SEQUENCE LISTING (8 sequences)

SEQ ID NO: 1 - DNA, 2470 bp, homo sapiens
ttgctcttggcagtgagcgtgaatgaatggcacagctcagcccctgaagcctgtgtgcagagattgagggattgtgatggagtagttcattcatgctcatgttaaggggggtgctaatagcagactagtgctcctgcgattattaatatctaggtctgggacagattgtgatggcttcttttccagttgccacctcagcagaaagggaaatagaaaaccctaacttgtaaagttagacaattagactgtaaagtttgtatatgtgacaacttcagatacaaagacacacacttacccttgacggggcttaagaggagagtgtcaaacataataccaaagtgaaagaagatagctcttcatctacaaattatttttaaacacatttaccaggttaaacaataactaatttttcggaagagaagagtacccaaagtcaaaatgccctaagacgaagagatgcttatggcatttttttttaaattaacaaaatgcaaagttagagtggttctgaaggaacctaggatgaataaggtacagacatgattattctaatggtgcagacaggattgagagagaaggggggaggggagagatggagaaaggcatggatggaagatgacgtttggattcagattttggaaaggagagtaaaggaaggaggtaagcagagatttattttttaaattttattaatgtgttttcccctctttttcttgttatttttctcatctgtctgttcatacttggatattttgtccaataaactatcttctaaggactctgaaaatgcactgaatatttttggagggtttactgggtgccagacgccactttaggagttttacatatcctctccatttcatttagttctcttagcacagagaagtgggagaagatagtcccattttacaggtgggatgaagagagagatggaggaatttgccccaggttactcagctagaaggtggtgaagaactcaagccttcggatatcagcgcctggcatttaactaccaatcggtcctgctgggactccggctcctctggcaccatccccgggacctactcagagagtttgcacgtggccggtcgcgttccatcgtctaacaaggtccagcacagcgcaaatccgaagatcgtctaccccggggaaaaagagagtctgtttaattctcctgtggccctccaagtgagttcttttgggttccattgcctagacgaggaaagtgaggctttgcctgctctgcgctcacagggtcggcaagtagtgggaccctaggttcctgcagtattccagagataatcaaagctgcacaggtctcgtcatttttatgcaaaggcgtccggaaggctcgaactctcccttgcacaagcccatctgtctctgtgcgccgcccccgggacacgaaagcagcggcgagcagcgccgagtgggtggagaaccgtcccccgccactcacccctcggccaactctccgcgccttctcagccggcacccacgaggccgacctctctcggcctaaaaaaaaaaaaaaaaaatcccggcctcccctgcaccccgcccgccgcccccagggagctgcattaatattaatctcgctgaataattgaaggccagagatttattcgagcttcggcgggggagggagcgcagctgggccgcgtttaggctgcaccacccgcgtgtttcagccgctcgactccgctggacctgggacccccagacgtgggaggatggggtgggtgtgcctgcctgtgagtttgggggtgagtgtgagctgaagcgggtgctccggggagtgaggagggagcgccaggggctgctccagggaggcggagacggaggggcatcccgggtctccgcgcggtcgcctgcgcttcaccccgcacggggtgacctggggccacgcgggcttcaggggaaacaatagctactccttagatcctgggctcctgccaccggcttgcccaagccttcccggacgagcggcggggcctcttttcttatttggctaatttatggcgagaggctggggggagggatggcagaggagggaccgcgactggagatgggggcggggggcggcggttaaaggagttgcccgaggcggcggcgcgggtgatgtcagctctcgacgaaaatagagagggatcgcctgcaaatccccagctccggcggggctaaaccttgcaatccctccctggccggcgccgagccagagagcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcg

SEQ ID NO: 2 - DNA, 164 bp, homo sapiens
tcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcg

SEQ ID NO: 3 - DNA, 370 bp, homo sapiens
cccgaggcggcggcgcgggtgatgtcagctctcgacgaaaatagagagggatcgcctgcaaatccccagctccggcggggctaaaccttgcaatccctccctggccggcgccgagccagagagcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcg

SEQ ID NO: 4 - DNA, 747 bp, homo sapiens
acgtgggaggatggggtgggtgtgcctgcctgtgagtttgggggtgagtgtgagctgaagcgggtgctccggggagtgaggagggagcgccaggggctgctccagggaggcggagacggaggggcatcccgggtctccgcgcggtcgcctgcgcttcaccccgcacggggtgacctggggccacgcgggcttcaggggaaacaatagctactccttagatcctgggctcctgccaccggcttgcccaagccttcccggacgagcggcggggcctcttttcttatttggctaatttatggcgagaggctggggggagggatggcagaggagggaccgcgactggagatgggggcggggggcggcggttaaaggagttgcccgaggcggcggcgcgggtgatgtcagctctcgacgaaaatagagagggatcgcctgcaaatccccagctccggcggggctaaaccttgcaatccctccctggccggcgccgagccagagagcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcg

SEQ ID NO: 5 - DNA, 998 bp, homo sapiens
ctccgcgccttctcagccggcacccacgaggccgacctctctcggcctaaaaaaaaaaaaaaaaaatcccggcctcccctgcaccccgcccgccgcccccagggagctgcattaatattaatctcgctgaataattgaaggccagagatttattcgagcttcggcgggggagggagcgcagctgggccgcgtttaggctgcaccacccgcgtgtttcagccgctcgactccgctggacctgggacccccagacgtgggaggatggggtgggtgtgcctgcctgtgagtttgggggtgagtgtgagctgaagcgggtgctccggggagtgaggagggagcgccaggggctgctccagggaggcggagacggaggggcatcccgggtctccgcgcggtcgcctgcgcttcaccccgcacggggtgacctggggccacgcgggcttcaggggaaacaatagctactccttagatcctgggctcctgccaccggcttgcccaagccttcccggacgagcggcggggcctcttttcttatttggctaatttatggcgagaggctggggggagggatggcagaggagggaccgcgactggagatgggggcggggggcggcggttaaaggagttgcccgaggcggcggcgcgggtgatgtcagctctcgacgaaaatagagagggatcgcctgcaaatccccagctccggcggggctaaaccttgcaatccctccctggccggcgccgagccagagagcagcggcctccaccgcctccccaggcgcgcacacacccgcacacgcgcacgcacgctcaccgtcctctgccaccactctctgctcccgccactcgccgcgcccgcgagccccgcagcaaagcacaggtggcagcggctgcaggggcgcatcgccggcgtgcgccctcctgcagccctgggcgcatcgctctctcggggaagccaccctcggagcccccggagctccccgccaagcgccatccccgcg

SEQ ID NO: 6 - DNA, 32 bp, homo sapiens
taatccgcgtcccggctctccacggcgcgcga

SEQ ID NO: 7 - DNA, 1700 bp, Homo sapiens
  CDS (275)...(895)
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtctgctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactca

SEQ ID NO: 8 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

(19) United States
(12) Patent Application Publication
(10) Pub. No.: US 2006/0127980 A1
(43) Pub. Date: Jun. 15, 2006

(54) Human excitatory amino acid transporter-2 gene promoter and uses thereof

(76) Inventors: Paul B. Fisher, Scarsdale, NY (US); Zhao-zhong Su, New York, NY (US)

Correspondence Address: BAKER & BOTTS, 30 ROCKEFELLER PLAZA, NEW YORK, NY, 10112, US

(21) Appl. No.: 11/197,226

(22) Filed: Aug. 4, 2005

Related U.S. Application Data
(63) Continuation of application No. PCT/US04/03700, filed on Feb. 9, 2004.
Application No. PCT/US04/03700 is a continuation-in-part of application No. 10/360,349, filed on Feb. 7, 2003.

(51) Int. Cl. A61K 38/17; C07K 14/705; C07H 21/04; C12N 5/08; C12P 21/06

(52) U.S. Cl. 435/69.1; 514/12; 435/320.1; 435/368; 530/350; 536/23.5

(57) ABSTRACT

The nucleic acid sequence of the human Excitatory Amino Acid Transporter-2 Gene (hEAAT2) promoter, a nucleic acid sequence that hybridizes to the hEAAT2 promoter nucleic acid sequence under stringent hybridization conditions, and a nucleic acid sequence that is functionally equivalent to the hEAAT2 promoter sequence are provided, as are vectors containing these nucleic acid sequences. In addition, methods for the use of these nucleic acids to achieve tissue- or cell-specific gene expression are provided, as are methods for the use of these hEAAT2 promoter nucleic acids to identify agents that can modulate glutamate transport or the activity of the glutamate promoter. Such agents may be useful in the prevention, palliation or treatment of neurodegenerative and/or cerebrovascular diseases.

The subject matter described herein was supported in part by National Institutes of Health Grant 5P01NS031492, so that the United States Government has certain rights herein.

### 1. INTRODUCTION

The present invention relates to nucleic acids comprising the promoter of the human Excitatory Amino Acid Transporter-2 (hEAAT2) Gene and related molecules, the use of these nucleic acids to achieve tissue- or cell-specific gene expression, and the use of these nucleic acids to identify agents that can modulate glutamate transport. Such agents may be useful in the prevention, palliation or treatment of neurodegenerative and/or cerebrovascular diseases.

### 2. BACKGROUND OF THE INVENTION

### 2.1 Control of Glutamate Levels in the CNS

The amino acid glutamate is the major excitatory neurotransmitter in the mammalian central nervous system (“CNS”; Robinson, 1998, Neurochem Int 33(6):479-491). Although essential for normal neuronal function and neurotransmission, accumulation of glutamate in the extracellular fluid of the CNS can cause neuronal damage and brain injury, a phenomenon termed “excitotoxicity” (Nicholls and Attwell, 1990. Trends Pharmacol Sci 11(11):462-468; Lipton and Rosenberg, 1994, N Engl J Med 330(9):613-622). It is well established that the concentration of extracellular glutamate in the CNS is controlled by Na⁺-dependent transport systems present in astrocytes and neurons, and that glutamate taken up by astrocytes is subsequently metabolized by glutamine synthase (Robinson, 1998, Neurochem Int 33(6):479-491). Thus, glutamate transport represents an important mechanism for maintaining low levels of this neurotransmitter in the extracellular milieu to promote synaptic signaling and to avoid glutamate-mediated excitotoxicity (Lipton and Rosenberg, 1994, N Engl J Med 330(9):613-622; Robinson, 1998, Neurochem Int 33(6):479-491).

Five cDNAs encoding excitatory amino acid transporters have been identified and cloned (EAAT1-5) (Arriza et al., 1994, J Neurosci 14(9):5559-5569; Fairman et al., 1995, Nature 375(6532):599-603; Arriza et al., 1997, Proc Natl Acad Sci USA 94(8):4155-4160). Among these five, EAAT1, also known as GLAST (Arriza et al., 1994, J Neurosci 14(9):5559-5569; Fairman et al., 1995, Nature 375(6532):599-603; Arriza et al., 1997, Proc Natl Acad Sci USA 94(8):4155-4160), and EAAT2, also referred to in the rodent as glutamate transporter-1 (GLT-1), are the major glutamate transporters in the CNS (Tanaka et al., 1997, Science 276(5319):1699-1702).

Astrocytes are the major cell type of the brain that expresses EAAT2, although neuronal expression has also been documented (as will be discussed below). Traditionally, the astrocyte was considered a minor player in neuronal function and in directing overall activities in the brain, providing only a maintenance role in brain homeostasis (Nicholls and Attwell, 1990, Trends Pharmacol Sci 11(11):462-468; Anderson et al., 2000, Glia 32(1):1-14). However, recent studies challenge these assumptions and suggest that, rather than being an innocuous bystander, the astrocyte may play a crucial role in regulating neuronal activity and signal transmission, and that deficiencies in these functions may contribute to neurodegeneration (Trotti et al., 1999, Nat Neurosci 2(5):427-433; Anderson et al., 2000, Glia 32(1):1-14; Carmignoto, 2000, Prog Neurobiol 62(6):561-581; Haydon, 2001, Nat Rev Neurosci 2(3):185-193).

One way astrocytes wield their effects on neuronal function is through the hEAAT2 transporter and its capacity to maintain stimulatory but non-toxic levels of free intrasynaptic L-glutamate in the area adjacent to neurons (Nicholls and Attwell, 1990, Trends Pharmacol Sci 11(11):462-468; Lipton and Rosenberg, 1994, N Engl J Med 330(9):613-622; Robinson, 1998, Neurochem Int 33(6):479-491). Abnormalities in this process result in the accumulation of excitotoxic levels of extracellular glutamate in synaptic clefts, leading to neuronal cell death (Nicholls and Attwell, 1990, Trends Pharmacol Sci 11(11):462-468; Lipton and Rosenberg, 1994, N Engl J Med 330(9):613-622; Robinson, 1998, Neurochem Int 33(6):479-491). Additional functions of astrocytes include stimulation of the number of synapses and an enhancement of synaptic efficiency by altering pre- and post-synaptic functions (Oliet et al., 2001, Science 292(5518):923-926; Ullian et al., 2001, Science 291(5504):657-661).

Astrocytes also display several excitatory features similar to those found in neurons, including the presence of functional neuronal nicotinic acetylcholine receptors (nACHRs) and Ca⁺⁺-dependent glutamate release (Iino et al., 2001, Science 292(5518):926-929; Sharma and Vijayaraghavan, 2001, Proc Natl Acad Sci USA 98(7):4148-4153; Ullian et al., 2001, Science 291(5504):657-661). These traits permit intracellular signaling between astrocytes and neurons and may even modulate neuronal signal transmission (Iino et al., 2001, Science 292(5518):926-929; Sharma and Vijayaraghavan, 2001, Proc Natl Acad Sci USA 98(7):4148-4153; Ullian et al., 2001, Science 291(5504):657-661).

Studies designed to elucidate the biochemical processes regulating glutamate transport have focused on rat astrocytes as a model system (Gegelashvili et al., 1997, J Neurochem 69(6):2612-2615; Schlag et al., 1998, Mol Pharmacol 53(3):355-369; Zelenaia et al., 2000, Mol Pharmacol 57(4):667-678). These investigations indicate that multiple and converging signal transduction pathways affecting astrocyte maturation regulate rodent GLT-1 expression, as monitored by changes in mRNA and protein levels, and consequently glutamate transport (Gegelashvili et al., 1997, J Neurochem 69(6):2612-2615; Schlag et al., 1998, Mol Pharmacol 53(3):355-369; Zelenaia et al., 2000, Mol Pharmacol 57(4):667-678).

Although the EAAT2 protein is predominantly expressed in astrocytes, expression of EAAT2 has also been observed in neurons during development (Yamada et al., 1998, J Neurosci 18(15):5706-5713), in response to ischemic insult (Martin et al., 1997, Ann Neurol 42(3):335-348), and in neurons grown in culture (Brooks-Kayal et al., 1998, Neurochem Int 33(2):95-100). At present, the neuroanatomical sites of EAAT2 expression are unresolved. Two semi-quantitative studies suggest uniform expression with minimal variations in different brain regions (Rothstein et al., 1994, Neuron 13(3):713-725; Robinson, 1998, Neurochem Int 33(6):479-491) while others suggest greater expression (8- to 10-fold) in the forebrain relative to the cerebellum (Lehre et al., 1995, J Neurosci 15(3 Pt 1):1835-1853; Milton et al., 1997, Brain Res Mol Brain Res 52(1):17-31).

### 2.2 Glutamate Excitotoxicity and Neurologic Disease

Reductions in EAAT2 protein expression have been correlated with neuropathology resulting from (i) ischemia (Torp et al., 1995, Exp Brain Res 103(1):51-58), (ii) temporal lobe epilepsy (Mathern et al., 1999, Neurology 52(3):453-472), (iii) Alzheimer's disease (Li et al., 1997, J Neuropathol Exp Neurol 56(8):901-911), (iv) Huntington's disease (Lipton and Rosenberg, 1994, N Engl J Med 330(9):613-622), and (v) amyotrophic lateral sclerosis (Bruijn et al., 1997, Neuron 18(2):327-338; Lin et al., 1998, Neuron 20(3):589-602). Glutamate excitotoxicity also has been implicated in numerous other CNS abnormalities, including pathological changes associated with head trauma, and in the immune-mediated damage present in multiple sclerosis (Smith et al., 2000, Nat Med 6(1):62-66). Further, a potential role has been proposed for astrocyte glutamate transport in HIV-1 related dementia (HAD) (Kaul et al., 2001, Nature 410(6831):988-994). In addition, malignant gliomas secrete glutamate, and it has been proposed that the resulting extracellular glutamate may contribute to tumor expansion (Takano et al., 2001, Nat Med 7(9):1010-1015). These findings emphasize the importance of glutamate transport and the EAAT2 transporter of astrocytes to normal brain function and their association with multiple pathologic changes in the brain.

Treatment strategies for disorders of glutamate transport and the neuronal excitotoxicity inherent therein have hitherto focused on treatment modalities collectively referred to as neuroprotectors (NPs). NPs are drugs, hormones, or other factors that reduce glutamate-mediated excitotoxicity, oppose the excessive release of glutamate, or block the intracellular effects of glutamate. NPs also include trophic factors that, through their direct effects on neuron growth and survival, may prevent or reverse the neurodegeneration that is often secondary to glutamate toxicity. At least 800 clinical trials of NPs are currently underway worldwide, and many more are contemplated. The most clinically-promising NP subgroups are antagonists for the N-methyl D-aspartate (NMDA) and amino-hydroxy-methyl-isoxalone propionic acid (AMPA) receptors, agonists for gamma-amino butyric acid (GABA) receptors, agents that promote the sequestration of intracellular Ca⁺⁺, inhibitors of nitric oxide (NO) modulation pathways, scavengers of free radicals, antagonists of sodium channels, inhibitors of glutamate release, activators of potassium channels, neurotrophic factors, and neuron replacement therapy.

Many of these NPs, such as NMDA or AMPA receptor antagonists, are small molecules that may prevent the excitatory effects of glutamate locally in the desired target region, but which may also interfere with glutaminergic signaling at distal sites, thereby altering desirable and physiologically-necessary processes unrelated to disease. Other treatments, such as the application of neurotrophic factors or the implantation of neurons or neuronal precursors, may act to restore neuronal cell mass lost through the degenerative process, but may not successfully recreate the synaptic connections destroyed by these processes. Thus, there is a strong and continuing need for the development of better treatments for diseases caused by glutamate excitotoxicity.

In accordance with the present invention, the promoter of the hEAAT2 has been identified and characterized. Moreover, the regulation of the activity of this promoter in response to a variety of intracellular-and extracellular signals has been determined. These findings, which are further described herein, indicate that the hEAAT2 promoter and related nucleic acids may be useful for the identification and development of novel agents for the regulation of the hEAAT2 promoter, and hence for the treatment of diseases caused by glutamate excitotoxicity.

### 3. SUMMARY OF THE INVENTION

The invention provides for isolated nucleic acids comprising a human Excitatory Amino Acid Transporter-2 Gene (hEAAT2) promoter, including nucleic acid molecules as depicted in FIG. 7 (SEQ ID NO:1), nucleic acids that hybridize to the hEAAT2 promoter nucleic acid sequence having SEQ ID NO:1 under stringent hybridization conditions, and nucleic acids that are homologous and functionally equivalent to the hEAAT2 promoter, collectively referred to as hEAAT2 promoter nucleic acids. Also provided are vectors comprising hEAAT2 promoter nucleic acids and cells comprising such vectors.

The invention further provides for met hods of achieving tissue- or cell-specific expression of a gene of interest comprising operatively linking a hEAAT2 promoter nucleic acid to the desired gene of interest, and introducing the resulting expression cassette into a target cell or tissue wherein cell- or tissue-specific gene expression is desired.

The invention further provides methods for the use of these nucleic acids to identify agents that can modulate glutamate transport comprising (i) operably linking an hEAAT2 promoter nucleic acid to a reporter gene of interest to form an expression cassette, (ii) introducing the resulting expression cassette into a target cell, (iii) contacting the target cell with a test agent, and (iv) comparing the level of reporter gene expression in the presence and absence of the test agent, wherein a test agent that modulates glutamate transport produces a discernible change in the level of reporter gene expression. Agents that increase promoter activity identified by this assay may be useful in the prevention, palliation or treatment of neurodegenerative and/or cerebrovascular diseases associated with glutamate excitotoxicity.

As shown in FIG. 6, many cellular processes interact to alter the transcriptional activity of the hEAAT2 promoter. Thus, the hEAAT2 promoter nucleic acids of the instant invention may be used as to identify test agents that affect these manifold processes, including agents that are agonists or antagonists of the epidermal growth factor (EGF), transforming growth factor-α (TGF-α) or tumor necrosis factor-α (TNF-α) receptors, agents that affect cellular levels of the EGF, TGF-α, or TNF-α receptors, or agents that modulate intracellular levels of cyclic adenosine monophosphate (cAMP), phosphoinositide-3 kinase (PI-3K); protein kinase C (PKC), protein kinase B (Akt), the TNF receptor-1 associated death domain protein (TRADD), TNF receptor associated factor 2 (TRAF2), nuclear factor kappa B-induced kinase (NIK), I-kappa B kinase (IKK), inhibitor of NF-κB (IκB), nuclear factor kappa B (NF-κB), protein kinase A (PKA), mitogen-activated protein kinase (MAPK), extracellular signal-regulated kinase (ERK), or the ras oncogene protein.

The present invention is also based, in part, on the discovery that the hEAAT2 promoter is highly active in astrocytes relative to other neuronal cell types, but is also active in neurons at various time points during the course of development of the nervous system and in response to ischemic insults of the nervous system. hEAAT2 also is down regulated as a function of neuropathology in ischemia, temporal lobe epilepsy, Alzheimer's disease (AD), Huntington's disease (HD), and amyotrophic lateral sclerosis (ALS). Accordingly, modulation of the hEAAT2 promoter via the agents identified by the instant invention may be used in the clinical management of these conditions.

### 3.1. Definitions

As used herein, the term “cDNA” can refer to a single-stranded or double-stranded DNA molecule. For a single-stranded cDNA molecule, the DNA strand is complementary to the messenger RNA (“mRNA”) transcribed from a gene. For a double-stranded cDNA molecule, one DNA strand is complementary to the mRNA and the other is complementary to the first DNA strand.

As used herein, a “coding sequence” or a “nucleotide sequence encoding” a particular protein is a nucleic acid molecule which is transcribed and translated into a polypeptide in vivo or in vitro when placed under the control of appropriate regulatory sequences. The boundaries of the coding sequence are determined by a start codon at the 5′-terminus and a translation stop codon at the 3′-terminus. A coding sequence can include, but is not limited to, prokaryotic nucleic acid molecules, cDNA from eukaryotic mRNA, genomic DNA from eukaryotic (e.g. mammalian) sources, viral RNA or DNA, and even synthetic nucleotide molecules. A transcription termination sequence will usually be located 3′ to the coding sequence.

As used herein, the term “control sequences” refers collectively to promoter sequences, polyadenylation signals, transcription termination sequences, upstream regulatory domains, enhancers and the like, and untranslated regions (UTRs) including 5′-UTRs and 3′-UTRs, which collectively provide for the transcription and translation of a coding sequence in a host cell.

As used herein, a control sequence “directs the transcription” of a coding sequence in a cell when RNA polymerase will bind the promoter sequence and transcribe the coding sequence into mRNA, which is then translated into the polypeptide encoded by the coding sequence.

As used herein, the term “gene” refers to a DNA molecule that either directly or indirectly encodes a nucleic acid or protein product that has a defined biological activity. One class of genes often encountered in the art is the so-called “reporter gene.” A reporter gene is any gene whose expression is used as a measure of the activity of the control sequences to which it is operably linked. Examples of commonly used reporter genes include, but are not limited to, a β-galactosidase gene, a chloramphenicol aminotransferase (CAT) gene, a luciferase (luc) gene, and genes encoding fluorescent proteins such as Green Fluorescent Protein (GFP), Blue Fluorescent Protein (BFP), etc. Ideally, reporter genes do not interfere with the underlying biological processes that are the target of the study. However, in some instances, it may be desirable to measure the activity of the control sequences by linking them to a gene whose product does alter the underlying biology of the system in which gene expression is occurring. Such genes, while also reporter genes, are often referred to as “biologically active” genes.

As used herein, the term “genomic DNA” refers to a DNA molecule from which an RNA molecule is transcribed. The RNA molecule is most often a messenger RNA (mRNA) molecule, which is ultimately translated into a protein that has a defined biological activity, but alternatively may be a transfer RNA (tRNA) or a ribosomal RNA (rRNA) molecule, which are mediators of the process of protein synthesis.

As used herein, two nucleic acid molecules are “functionally equivalent” when they share two or more quantifiable biological functions. For example, nucleic acid molecules of different primary sequence may encode identical polypeptides; such molecules, while distinct, are functionally equivalent. In this example, these molecules will also share a high degree of sequence homology. Similarly, nucleic acid molecules of different primary sequence may share activity as a promoter of RNA transcription, wherein said RNA transcription occurs in a specific subpopulation of cells, and responds to a unique group of regulatory substances; such nucleic acid molecules are also functionally equivalent. Provided with the teachings included herein, especially those of FIGS. 2-5, one of ordinary skill in the art would be able to identify nucleic acid molecules that are functionally equivalent to the hEAAT2 promoter. For example, a nucleic acid molecule is “functionally equivalent” to the hEAAT2 nucleic acid sequence depicted in FIG. 7 if it is (i) approximately ten-fold more active as a promoter of transcription in primary human fetal astrocytes than in human mammary epithelial cells (HMEC), human prostate epithelial cells (HPEC), or any of the other cell types analyzed in FIG. 2, AND (ii) this promoter activity is either enhanced by exposure to cyclic adenosine monophosphate (cAMP) or attenuated by exposure to tumor necrosis factor-α (TNF-α).

As used herein, a “heterologous” region of a DNA construct is an identifiable segment of DNA within or attached to another DNA molecule that is not found in association with the other molecule in nature. An example of a heterologous coding sequence is a construct where the coding sequence itself is not found in nature (e.g. synthetic sequences having codons different from the native gene). Allelic variation or naturally occurring mutational events do not give rise to a heterologous region of DNA as used herein.

As used herein, two nucleic acid molecules are “homologous” when at least about 60% to 75% or preferably at least about 80% or most preferably at least about 90% of the nucleotides comprising the nucleic acid molecule are identical over a defined length of the molecule, as determined using standard sequence analysis software such as Vector NTI, GCG, or BLAST. DNA sequences that are homologous can be identified by hybridization under stringent conditions, as defined for the particular system. Defining appropriate hybridization conditions is within the skill of the art. See e.g. Current Protocols in Molecular Biology, Volume I. Ausubel et al., eds. John Wiley: New York N.Y., pp. 2.10.1-2.10.16, first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised of 40 mM NaPO₄, pH 7.2, 1-2% SDS and 1 mM EDTA. Again, a washing temperature of at least 65-68° C. is recommended, but the optimal temperature required for a truly stringent wash will depend on the length of the nucleic acid probe, its GC content, the concentration of monovalent cations and the percentage of formamide, if any, that was contained in the hybridization solution (Current Protocols in Molecular Biology, Volume I. Ausubel et al., eds. John Wiley: New York N.Y., pp. 2.10.1-2.10.16. 1989 with annual updating).

As used herein, the term “nucleic acid molecule” includes both DNA and RNA and, unless otherwise specified, includes both double-stranded and single-stranded nucleic acids. Also included are molecules comprising both DNA and RNA, either DNA/RNA heteroduplexes, also known as DNA/RNA hybrids, or chimeric molecules containing both DNA and RNA in the same strand. Nucleic acid molecules of the invention may contain modified bases. The present invention provides for nucleic acid molecules in both the “sense” orientation (i.e. in the same orientation as the coding strand of the gene) and in the “antisense” orientation (i.e. in an orientation complementary to the coding strand of the gene).

As used herein, the term “operably linked” refers to an arrangement of nucleic acid molecules wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

As used herein, the term “sequence” refers to a nucleic acid molecule having a particular arrangement of nucleotides, e.g. the hEAAT2 promoter sequence shown in FIG. 7 (SEQ ID NO:1), or a particular function, e.g. a termination sequence. Where specified, the term “sequence” refers specifically to the order of nucleotides, e.g. the sequence of the hEAAT2 promoter as set forth in SEQ ID NO: 1.

As used herein, exogenous DNA may be introduced into a cell by processes referred to as “transduction”, “transfection,” or “transformation.” Transduction refers to the introduction of genetic material, either RNA or DNA, across the membrane of a eukaryotic cell via a vector derived from a virus. Transfection refers to the introduction of genetic material across the membrane of a eukaryotic cell by chemical means such as by calcium phosphate-mediated precipitation, by mechanical means such as electroporation, or by physical means such as bioballistic delivery. Transformation refers to the introduction of genetic material into non-eukaryotic cells, such as bacterial cells or yeast cells, by chemical, mechanical, physical or biological means. The genetic material delivered into the cell may or may not be integrated (covalently linked) into chromosomal DNA. For example, the genetic material may be maintained on an episomal element, such as a plasmid. A stably transformed non-eukaryotic cell or stably transfected eukaryotic cell is generally one in which the exogenous DNA has become integrated into the chromosome so that it is inherited by daughter cells through chromosome replication, or one which includes stably-maintained extrachromosomal plasmids. This stability is demonstrated by the ability of the cell to establish clones comprised of a population of daughter cells containing the exogenous DNA. Cells containing exogenous DNA that is not integrated into the chromosome or maintained extrachromosomally through successive generations of progeny cells are said to be “transiently transformed” or “transiently transfected.”

As used herein and according to scientific convention, the italicized form of “hEAAT2” (i.e. “hEAAT2”) will be used when referring to the hEAAT2 gene or its promoter, while the non-italicized form of “hEAAT2”(i.e. “hEAAT2”) will be used when referring to the hEAAT2 protein.

### 4. DESCRIPTION OF THE FIGURES

FIG. 1A-C. A. Schematic of the hEAAT2 gene and its promoter. Exons are indicated by number above the bold boxes. The locations of various transcription factor-binding sites in the hEAAT2 gene promoter are indicated by the various rectangles in the lower portion of the panel. The numbers above the rectangles provide the approximate location of these binding sites within the hEAAT2 gene promoter. B. Primer extension analysis of the hEAAT2 gene. Lanes 1 to 3 contain different concentrations of labeled probe, 1: 10⁴ cpm; 2: ˜10⁵ cpm; 3: ˜5×10⁴ cpm. C. Intron-exon structure of the hEAAT2 gene. The sizes of the exons and introns of the hEAAT2 gene are indicated.

FIG. 2A-B. A. Relative expression of the hEAAT2 promoter in PHFA and various normal and tumor cell lines. B. Effect of passage number on flhEAAT2Prom-luc activity in PHFA cells.

FIG. 3A-B. Deletion analysis of the hEAAT2 promoter. A. 5′-deletions of the hEAAT2 promoter. B. Relative fold luciferase activity of the various promoter deletion constructs in PHFA.

FIG. 4A-D. Effect of modulators of hEAAT2 activity on hEAAT2 expression and hEAAT2 promoter activity in PHFA. A. A Northern analysis of hEAAT2 and GAPDH mRNA expression following treatment with EGF (30 ng/ml), TGF-α (30 ng/ml), dbcAMP (200 μM, bromo-cAMP (100 μM) or TNF-α (200 U/ml) for 7 days. B. Nuclear run-on assays determining the relative rates of hEAAT2 and GAPDH transcription as a function of 7-day treatment with the same concentrations of the indicated agents. C. A time course of mRNA expression of hEAAT2 and GAPDH following treatment with EGF, bromo-cAMP or TNF-α (same concentrations as in A.). D. Fold luciferase activity in PHFA transfected with flhEAAT2Prom-luc or various deletion mutants of hEAAT2 and non-transfected controls treated with the indicated compounds for four days.

FIG. 5A-E. Effect of pharmacological inhibitors on hEAAT2 promoter activity, and mRNA and protein expression in PHFA following various treatment protocols. A. hEAAT2 promoter activity in PHFA, either untreated (−) or treated with EGF (30 ng/ml), in the absence (−) or presence (+) of KT5720 (5 μM), AG1478 (1 μM), PDTC (100 μM), wortmannin (WRT) (100 nM) or PD98049 (PD) (50 μM). B. hEAAT2 promoter activity in PHFA, either untreated (−) or treated with bromo-cAMP (250 μM) in the absence (−) or presence (+) of KT5720 (5 μM), AG1478 (1 μM), PDTC (100 μM), wortmannin (WRT) (100 nM) or PD98049 (PD) (50 μM). C. hEAAT2 promoter activity in PHFA, either untreated (−) or treated with TNF-α (200 U/ml), in the absence (−) or presence (+) of KT5720 (5 μM), AG1478 (1 μM), PDTC (100 μM), wortmannin (WRT) (100 nM) or PD98049 (PD) (50 μM). D. The effect of the various treatment protocols on hEAAT2 and GAPDH mRNA levels. Upper panel presents relative hEAAT2 RNA expression versus GAPDH expression relative to control untreated or treated samples based on scanning of autoradiograms. Lower panel, actual Northern blots. E. The effect of the various treatment protocols on EAAT2 and ACTIN protein levels. Upper panel presents relative EAAT2 protein expression versus ACTIN expression relative to control untreated or treated samples based on scanning of autoradiograms. Individual experiments were performed 3 times using triplicate samples and S.D. from the mean is presented.

FIG. 6. Schematic of pathways and inhibitors effecting hEAAT2 promoter activity. Abbreviations: EGF, Epidermal Growth Factor; EGF-R, Epidermal Growth Factor Receptor; PI-3K, Phosphoinositide-3 Kinase; PKC, Protein Kinase C; Akt, protein kinase B; TNF-α, Tumor Necrosis Factor-α; TNFR, Tumor Necrosis Factor-α Receptor; TRADD, TNF receptor-1 Associated Death Domain Protein; TRAF2, TNF Receptor Associated Factor 2; NIK, Nuclear Factor kappa B-induced Kinase; IKK, I-Kappa B Kinase; IκB, Inhibitor of NF-κB; PDTC, Pyrrolidine Dithiocarbamate; NF-κB, Nuclear Factor kappa B; cAMP, cyclic Adenosine Monophosphate; PKA, Protein Kinase A; MAPK, Mitogen-activated Protein Kinase; ERK, Extracellular Signal-regulated Kinase.

FIG. 7. Nucleic acid sequence of the hEAAT2 promoter (SEQ ID NO:1).

FIG. 8. Schematic representation of an expression cassette in which transcription of a reporter gene is regulated by the hEAAT2 promoter.

FIG. 9. Schematic representation of an expression cassette in which transcription of a therapeutic gene is regulated by the hEAAT2 promoter.

FIG. 10. Effect of various agents on hEAAT2 promoter activity on primary human fetal astrocytes (PHFA). A: ceftriaxone; B: amoxicillin; C: chloramphenicol; D: thiamphenicol; E: vancomycin; F: glycine; G: glutamate; H: DMSO; I: dibutyryl cAMP. PHFA (passage #3) were seeded at 1×10⁵ cells/35-mm plate. Twenty-four h later cells were untreated (CON) or received the indicated compound at a final concentration of 10 μM. Forty-eight h later the cells were transfected with a FL pGL3/EAAT2 luciferase reporter construct (5 μg) plus a pSVβGalactosidase construct (1 μg) using the calcium phosphate precipitation method (Su et al. 2003, Proc. Natl. Acad. Sci. USA 100:1955-1960). After an additional 48 h, cell lysates were prepared and luciferase activity was determined using the Luciferase Assay System Kit (Promega, E1501) and luminescence determined using a luminometer (Turner Designs, TD20/20) (Su et al. 2003, Proc. Natl. Acad. Sci. USA 100:1955-1960). Data presented are the average of 3 independent plates±S.D. Qualitatively similar results were obtained in 2 additional experiments.

FIG. 11. Effect of various agents on hEAAT2 promoter activity in immortalized primary human fetal astrocytes (PHFA-Im). A: ceftriaxone; B: amoxicillin; C: chloramphenicol; D: thiamphenicol; E: vancomycin; F: glycine; G: glutamate; H: DMSO; I: dibutyryl cAMP. PHFA-Im is a human telomerase-immortalized clone of primary human fetal astrocytes. PHFA-Im cells were seeded at 5×10⁴ cells/35-mm plate. Twenty-four h later cells were untreated (CON) or received the indicated compound at a final concentration of 10 μM. Forty-eight h later the cells were transfected with a FL pGL3/EAAT2 luciferase reporter construct (5 μg) plus a pSVβGalactosidase construct (1 μg) using the calcium phosphate precipitation method (Su et al. 2003, Proc. Natl. Acad. Sci. USA 100:1955-1960). After an additional 48 h, cell lysates were prepared and luciferase activity was determined using the Luciferase Assay System Kit (Promega, E1501) and luminescence determined using a luminometer (Turner Designs, TD20/20) (Su et al. 2003, Proc. Natl. Acad. Sci. USA 100:1955-1960). Data presented are the average of 3 independent plates±S.D. Qualitatively similar results were obtained in 2 additional experiments.

FIG. 12. Effect of various agents on hEAAT2 promoter activity in H4 human malignant glioma cells. A: ceftriaxone; B: amoxicillin; C: chloramphenicol; D: thiamphenicol; E: vancomycin; F: glycine; G: glutamate; H: DMSO; I: dibutyryl cAMP. H4 is a rare clone of malignant glioma cells that support hEAAT2 promoter activity. H4 cells were seeded at 5×10⁴ cells/35-mm plate. Twenty-four h later cells were untreated (CON) or received the indicated compound at a final concentration of 10 μM. Forty-eight h later the cells were transfected with a FL pGL3/EAAT2 luciferase reporter construct (5 μg) plus a pSVβGalactosidase construct (1 μg) using the calcium phosphate precipitation method (Su et al. 2003, Proc. Natl. Acad. Sci. USA 100:1955-1960). After an additional 48 h, cell lysates were prepared and luciferase activity was determined using the Luciferase Assay System Kit (Promega, E1501) and luminescence determined using a luminometer (Turner Designs, TD20/20) (Su et al. 2003, Proc. Natl. Acad. Sci. USA 100:1955-1960). Data presented are the average of 3 independent plates±S.D. Qualitatively similar results were obtained in 2 additional experiments.

### 5. DETAILED DESCRIPTION OF THE INVENTION

For clarity of presentation, and not by way of limitation, the detailed description of the invention is divided into the following subsections:

(1) hEAAT2 promoter nucleic acid molecules;

(2) hEAAT2 promoter expression cassettes;

(3) methods of identifying agents that modulate glutamate transport;

(4) methods of identifying agents that modulate signal transduction pathways or other biological processes that regulate extracellular glutamate levels; and

(5) hEAAT2 promoter/mda-7 constructs and their uses.

### 5.1 hEAAT2 Promoter Nucleic Acid Molecules

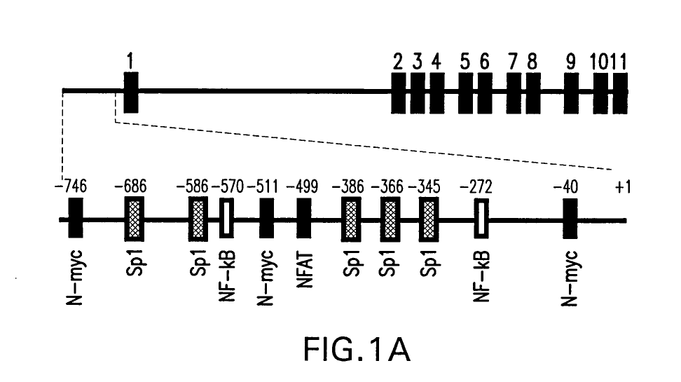

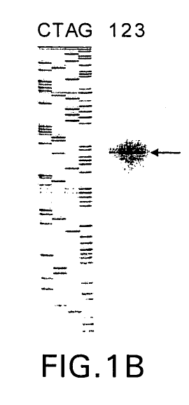

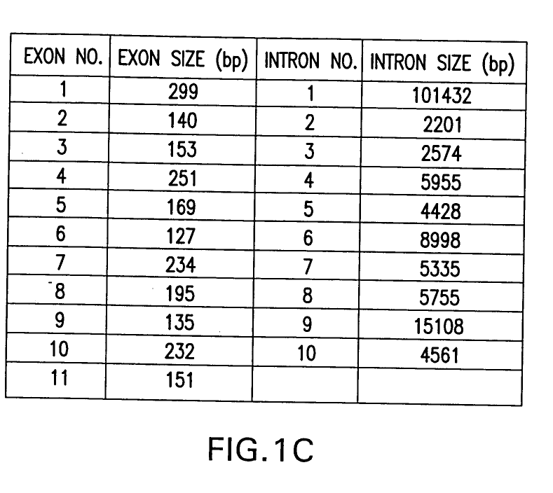

The present invention relates to compositions and/or methods which comprise and/or utilize, respectively, the various nucleic acid molecules that may be derived from the hEAAT2 gene promoter depicted schematically in FIG. 1. Nucleic acids may be DNA or RNA, and may comprise modified bases.

Thus, the invention provides for nucleic acid molecules including the following, taken singly or in combination, all of which are referred to herein as “hEAAT2 promoter nucleic acid molecules”:

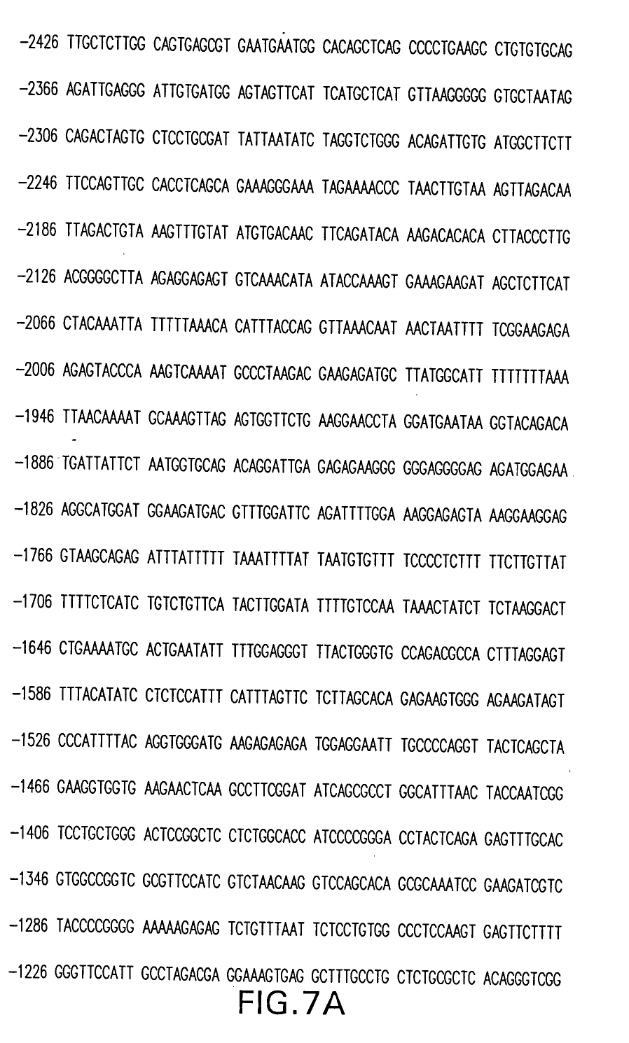

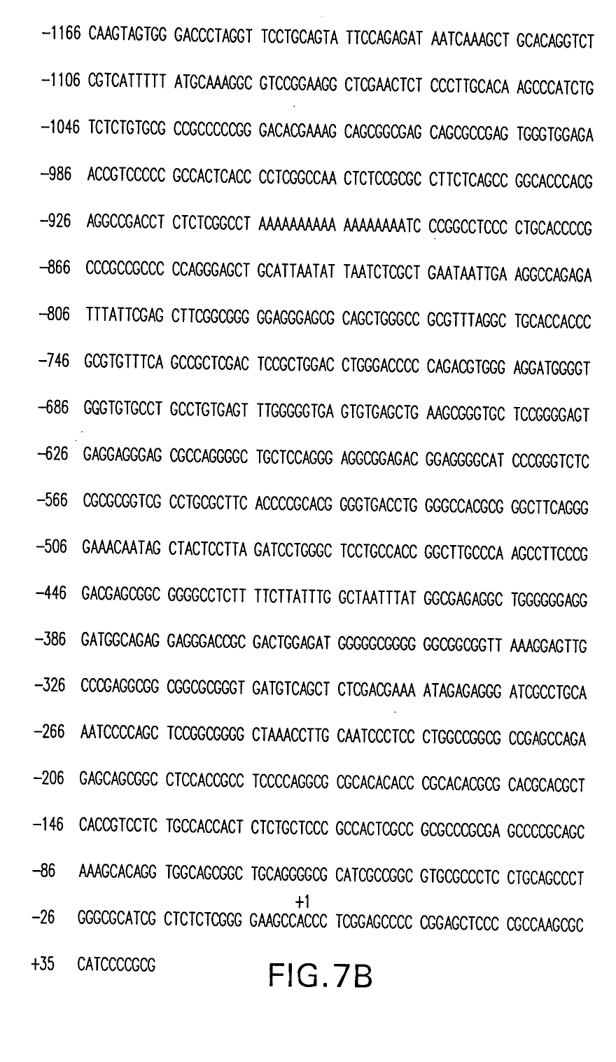

(i) nucleic acid molecules having sequences found immediately upstream of exon 1 of the hEAAT2 gene (e.g. the approximately 2.5 kb of nucleic acid sequence lying in the 5′ direction relative to exon 1) and that regulate the transcription of the hEAAT2 gene (i.e. the hEAAT2 promoter), especially those comprising at least a promoter-effective portion of the nucleic acid sequence set forth in FIG. 7 (nucleotide −2426 through nucleotide +44; SEQ ID NO:1), the 3′ portion of which is also depicted schematically in FIG. 1A;

(ii) nucleic acid molecules that specifically hybridize to the nucleic acids described above in (i); and

(iii) nucleic acid molecules that are homologous and functionally equivalent to the nucleic acids described above in (i).

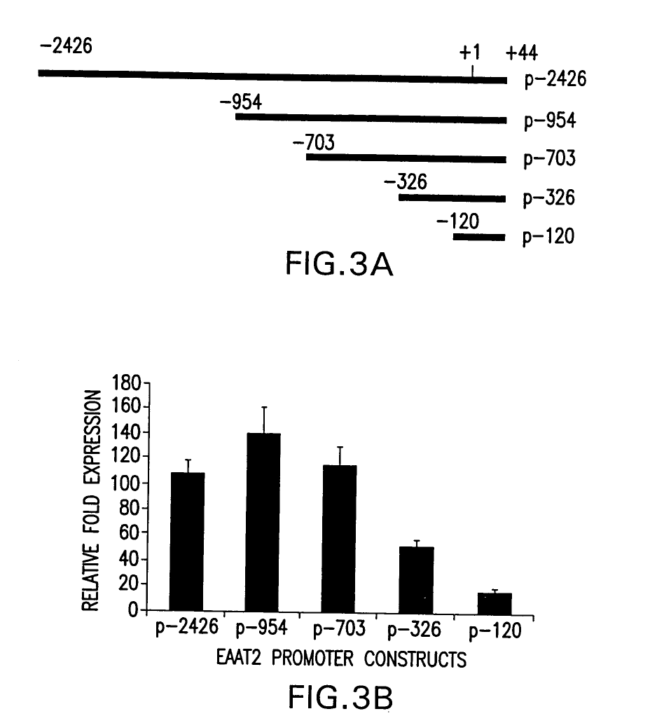

Each of the three foregoing classes of molecules is discussed in greater detail below. In a first set of embodiments, the present invention encompasses nucleic acid molecules spanning the region set forth in FIG. 7 (SEQ ID NO:1) or portions thereof, including but not limited to those depicted schematically in FIG. 3A. Preferably such molecules are between 50 and 2470 nucleotides in length, including, but not limited to, molecules which are between 50 and 500 nucleotides in length, and molecules which are between 500 and 2470 nucleotides in length. In preferred embodiments, such molecules are the nucleic acid molecules consisting essentially of (i) nucleotides −120 to +44 of the hEAAT2 gene (SEQ ID NO:2) or (ii) nucleotides −326 to +44 of the hEAAT2 gene (SEQ ID NO:3), both of which are depicted schematically in FIG. 3A. In more preferred embodiments, such molecules consisting essentially of nucleotides −703 to +44 of the hEAAT2 gene (SEQ ID NO:4) or nucleotides −954 to +44 of the hEAAT2 gene (SEQ ID NO:5), both of which are also depicted schematically in FIG. 3A. Based on the enzyme(s) utilized for its isolation and subcloning, the hEAAT2 promoter of the instant invention may, in certain embodiments, extend in the 5′ direction upstream of nucleotide −2426 and/or in the 3′ direction downstream of nucleotide +44. For example, but not by way of limitation, the 3′ terminal of SEQ ID NO:1 is created by the specific digestion of the sequence 5′-CCGCGG-3′ by the restriction endonuclease Sac II, which deletes the fragment 5′-GCGG-3′ from the 3′ end of the hEAAT2 promoter to yield SEQ ID NO:1. If another restriction endonuclease is used to isolate the hEAAT2 promoter, the 3′ end of the hEAAT2 promoter may comprise this 5′-GCGG-3′ fragment.

In a second set of embodiments, the present invention provides for nucleic acid molecules that hybridize to nucleic acid molecules encompassed in SEQ ID NO:1 (e.g. for use as probes or to silence promoter activity using antisense or triplex technologies) under stringent hybridization conditions. Defining appropriate hybridization conditions is within the skill of the art. See e.g. Current Protocols in Molecular Biology, Volume I. Ausubel et al., eds. John Wiley: New York N.Y., pp. 2.10.1-2.10.16, first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised of 40 mM NaPO₄, pH 7.2, 1-2% SDS and 1 mM EDTA. Again, a washing temperature of at least 65-68° C. is recommended, but the optimal temperature required for a truly stringent wash will depend on the length of the nucleic acid probe, its GC content, the concentration of monovalent cations and the percentage of formamide, if any, that was contained in the hybridization solution (Current Protocols in Molecular Biology, Volume I. Ausubel et al., eds. John Wiley: New York N.Y., pp. 2.10.1-2.10.16. 1989 with annual updating), all of which can be determined by the skilled artisan. Such molecules may have a range of sizes between 50 and 5000 nucleotides in length, but preferably between 50 and 2470 nucleotides in length. The washing conditions may be varied by alteration of temperature or salt concentration, so that the positively hybridizing molecules are at least 80% homologous, preferably 90% homologous, and most preferably 95% homologous to the target molecule. These molecules are not of a fixed or specified length, but their base pair compositions and lengths will be determined by their need to positively hybridize to the target sequences with at least the minimum degree of homology necessary to distinguish the target sequence from non-target sequences.

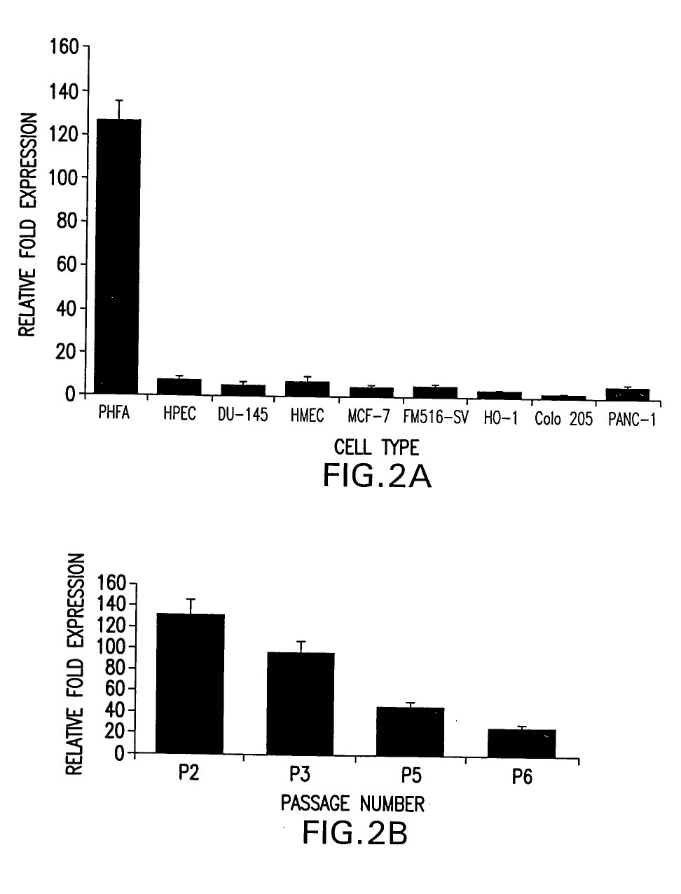

In a third set of embodiments, the present invention provides for nucleic acid molecules that are homologous and functionally equivalent to the foregoing molecules. In this context, two nucleic acid molecules are homologous when at least about 60% to 75% or preferably at least about 80% or most preferably at least about 90% of the nucleotides comprising the nucleic acid molecule are identical over a defined length of the molecule, as determined using standard sequence analysis software, and wherein the nucleic acids still qualitatively maintain the biological function associated with the nucleic acid sequence to which they are being compared. For example, a particular nucleic acid molecule is homologous and functionally equivalent to the hEAAT2 promoter nucleic acid sequence depicted in FIG. 7 (SEQ ID NO:1) if it is (i) at least 60% to 75% identical over the 2470 bp length of this sequence, (ii) approximately ten-fold more active as a promoter of transcription in primary human fetal than in human mammary epithelial cells (HMEC), human prostate epithelial cells (HPEC), or any of the other cell types analyzed in FIG. 2, AND (iii) its promoter activity is either enhanced by exposure to cyclic adenosine monophosphate (cAMP) or attenuated by exposure to tumor necrosis factor-α (TNF-α). Homologous nucleic acid sequences can be readily identified by various hybridization techniques known to those of ordinary skill in the art, including Southern hybridization as described above. Defining appropriate hybridization conditions to achieve a desired degree of homology between two nucleic acid molecules is well within the skill of the ordinary artisan. See e.g. Current Protocols in Molecular Biology, Volume I. Ausubel et al., eds. John Wiley: New York N.Y., pp. 2.10.1-2.10.16, first published in 1989 but with annual updating. Functionally equivalent nucleic acids can be readily determined by one of ordinary skill in the art based on the teachings provided herein, especially those of FIGS. 2-5.

### 5.2 hEAAT2 Promoter Expression Cassettes

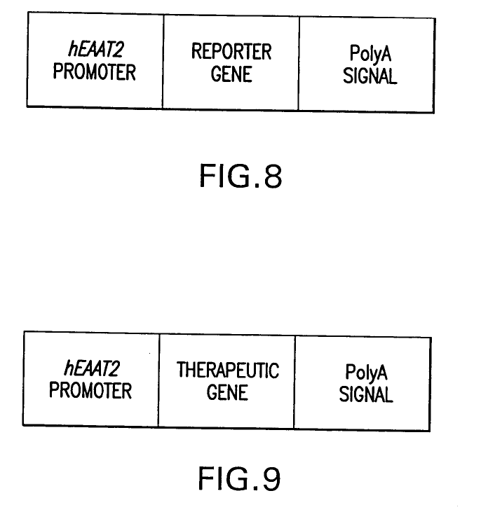

The present invention also provides for a hEAAT2 promoter expression cassette in which the coding region of a gene of interest is operably linked, on its 5′ end, to an hEAAT2 promoter nucleic acid molecule as described above and, on its 3′ end, by a polyadenylation (polyA) signal such that the coding region is under the transcriptional control of the hEAAT2 promoter nucleic acid molecule. The coding region contained within the hEAAT2 promoter expression cassette may comprise a physiologically-inert (i.e. a “reporter” gene; see FIG. 8), including but not limited to regions encoding chloramphenicol aminotransferase (CAT), luciferase (luc), β-galactosidase (β-gal), β-glucuronidase (β-gluc), β-lactamase (β-lac), alkaline phosphatase (AP), secreted alkaline phosphatase (SEAP), or a fluorescent protein, such as green fluorescent protein (GFP), blue fluorescent protein (BFP), etc.

Alternatively, the hEAAT2 promoter expression cassette may comprise a biologically-active gene, including but not limited to a pro- or anti-apoptotic gene, a suicide gene (such as oncogenes), a tumor suppressor gene, a gene encoding a receptor for a neurotransmitter or other extracellular ligand, a gene encoding an ion channel, a gene encoding a ribozyme, a gene encoding an oligonucleotide capable of acting as an antisense or triplex reagent for gene silencing or RNA interference, a gene encoding a toxin, a gene encoding a prodrug enzyme, a gene encoding a growth factor, or any other physiologically-relevant or therapeutically desirable genes known to those of ordinary skill in the art. See FIG. 9.

Non-limiting examples of pro-apoptotic or cytolytic gene products include a dominant negative Iκ-B, caspase-3, caspase-6, and a fusion protein containing a toxic moiety and the HSV VP22 protein.

Suicide genes encode proteins or agents that inhibit tumor cell growth or promotes tumor cell death. Suicide genes include but are not limited to genes encoding enzymes (e.g. prodrug enzymes), oncogenes, tumor suppressor genes, genes encoding toxins, genes encoding cytokines, growth factors, or a gene encoding oncostatin.

One purpose of the transgene can be to inhibit the growth of, or kill, a cancer cell or produce agents which directly or indirectly inhibit the growth of, or kill, a cancer cell.

Suitable prodrug enzymes include but are not limited to thymidine kinase (TK), human β-glucuronidase, xanthine-guanine phosphoribosyltransferase (GPT) or cytosine deaminase (CD) from E. coli, or hypoxanthine phosphoribosyl transferase (HPRT).

Examples of oncogenes and tumor suppressor genes include but are not limited to neu, EGF, ras (including H-, K-, and N-ras), p53, p16, p21, retinoblastoma tumor suppressor gene (Rb), the Wilm's Tumor gene, phosphotyrosine phosphatase (PTPase), and nm23.

Examples of suitable toxins include but are not limited to Pseudomonas exotoxins A and S; diphtheria toxin (DT); E. coli LT toxins, Shiga toxin, Shiga-like toxins (SLT-1, -2), ricin, abrin, supporin, and gelonin.

Suitable cytokines include but are not limited to interferons, interleukins, and tumor necrosis factor (TNF) (Horisberger et al., 1990, J. Virol. 64(3):1171-81; Ulich et al., 1991, J. Immunol. 146(7):2316-23; Breviario et al., 1992, J. Biol. Chem. 267(31):22190-7; Espinoza-Delgado et al., 1992, J. Immunol. 149(9):2961-8; Li et al., 1992, J. Immunol 148(3):788-94; Mauviel et al., 1992, J. Immunol. 149(9):2969-76; Martinez et al., 1993, Transplantation 55(5):1159-66; Pizarro et al., 1993, Transplantation 56(2):399-404; Wong et al., 1993, Science 228:810); WO93/23034; Algate et al., 1994, Blood 83(9):2459-68; Cluitmans et al., 1994, Ann. Hematol. 68(6):293-8; Lagoo et al., 1994, J. Immunol. 152(4):1641-52; and Pang et al., 1994, Clin. Exp. hnmunol. 96(3):437-43). Those of ordinary skill in the art ill recognize that the instant invention is amenable to use with a wide variety of cytokines, suicide genes, pro-apoptotic genes, or other genes whose products act to inhibit or suppress cell growth.

Growth factors suitable for use in the instant invention include transforming growth factor-α. (TGFα) and β (TGFβ), cytokine colony stimulating factors (Kay et al., 1991, J. Exp. Med. 173(3):775-8; Sprecher et al., 1992, Arch. Virol. 126(1-4):253-69; de Wit H et al., 1994, Br. J. Haematol. 86(2):259-64; and Shimane et al., 1994, BBRC 199(1):26-32), and a variety of neurotrophins including but not limited to nerve growth factor (NGF), beta nerve growth factor (BNGF), ciliary neurotrophic factor (CNTF), brain-derived neurotrophic factor (BDNF), and glial cell line-derived neurotrophic factor (GDNF).

The hEAAT2 promoter expression cassettes described herein may further comprise signal or secretory sequences to promote proteolytic processing, intracellular transport and extracellular secretion of peptides whose expression is regulated by the hEAAT2 gene promoter of the instant invention. Such signals are usually located at the 5′ end of the gene contained within the expression cassette, but may be placed in any location whereby processing and secretion of the synthesized protein is facilitated.

The hEAAT2 promoter expression cassettes may be incorporated into various vectors to facilitate their delivery into target cells, either in vitro or in vivo. Suitable expression vectors include nonvirus-based DNA or RNA delivery systems as well as virus-based vectors. Non-limiting examples of nonvirus-based vectors are plasmids, such as pcDNA3.1 (Invitrogen, San Diego, Calif.), etc., episomes such as pREP or pCEP (Invitrogen, San Diego, Calif.), etc., cosmids, or artificial chromosomes such as yeast artificial chromosomes (YACs) or bacterial artificial chromosomes (BACs). These nonvirus-based vectors may be delivered as so-called naked nucleic acids (Wolff et al., 1990, Science 247:1465-1468), nucleic acids encapsulated in liposomes (Nicolau et al., 1987, Methods in Enzymology 149:157-176), nucleic acid/lipid complexes (Legendre and Szoka, 1992, Pharmaceutical Research 9:1235-1242), and nucleic acid/protein complexes (Wu and Wu, 1991, Biother. 3:87-95). The nonvirus-based nucleic acid may be introduced into the cell by any standard technique, including transfection, transduction, electroporation, bioballistics, microinjection, etc.

Examples of appropriate virus-based gene transfer vectors include, but are not limited to, those derived from retroviruses, for example Moloney murine leukemia-virus based vectors such as LX, LNSX, LNCX or LXSN (Miller and Rosman, Biotechniques 1989;7:980-989; U.S. Pat. Nos. 6,025,192 and 6,255,071); lentiviruses, for example human immunodeficiency virus (“HIV”) (Case et al., 1999, Proc. Natl. Acad. Sci. U.S.A. 96: 22988-2993), feline leukemia virus (“FIV”) (Curran et al., 2000, Molecular Ther. 1:31-38) or equine infectious anemia virus (“EIAV”)-based vectors (Olsen, 1998, Gene Ther. 5:1481-1487); adenoviruses (Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806; Connelly, 1999, Curr. Opin. Mol. Ther. 1(5):565-572; Zhang, 1999, Cancer Gene Ther. 6(2):113-138;), for example AdS/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988,Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently human astrocytes, or other target cells as desired, and that can accommodate the particular hEAAT2 promoter expression cassette being examined.

Depending on the intended application, target cells for the hEAAT2 promoter expression cassette may include eukaryotic cells, bacteria, fungi (e.g. yeast), insect cells, etc. The instant invention therefore provides a cell, in preferred embodiments a mammalian cell, and in most preferred embodiments a human cell, comprising an hEAAT2 expression cassette in which an hEAAT2 nucleic acid, as defined above, is operably linked to a reporter gene. This cell may be provided by a variety of means, including the transformation or transient or stable transfection of the target cell by a plasmid comprising the hEAAT2 expression cassette, or by transduction of the target cell by a virus-based vector containing the hEAAT2 expression cassette. The artisan of ordinary skill will recognize the existence of many technological variations useful for the creation of a cell suitable for the purposes described herein.

The hEAAT2 promoter expression cassette described herein may be used to achieve cell- or tissue-specific expression of a given gene of interest. Such cell- or tissue-specific expression may be useful for scientific, diagnostic or therapeutic purposes. In non-limiting embodiments, such specific expression may be astrocyte-specific, neuron-specific or brain cell-specific.

### 5.3 Identification of Agents that Modulate Glutamate Transport

The present invention provides for the use of hEAAT2 promoter nucleic acids to identify agents that can modulate glutamate transport. These methods comprise (i) operably linking a nucleic acid sequence comprising an hEAAT2 promoter nucleic acid to a reporter gene of interest, (ii) introducing the resulting hEAAT2 promoter expression cassette into a target cell, (iii) contacting the target cell with a test agent that potentially modulates glutamate transport, and (iv) comparing the level of reporter gene expression in the presence and absence of the test agent, wherein a test agent that modulates glutamate transport is one that produces a discernible change in the level of reporter gene expression in the presence and absence of the agent. Agents that increase hEAAT2 promoter activity identified by this assay may be useful in the prevention, palliation or treatment of neurodegenerative and/or cerebrovascular diseases associated with glutamate excitotoxicity.

Cells useful for the assays of the present invention include any eukaryotic or prokaryotic cells in which the hEAAT2 promoter is active. Preferred cells include but are not limited to primary human fetal astrocytes (PHFA), PHFA cells immortalized by transformation with the human telomerase reverse transcriptase (hTERT), or H4 malignant human glioma cells. In a particular, non-limiting embodiment, the cells may be PHFA-Im cells, which are PHFA cells immortalized by transformation with hTERT, as deposited with ATCC and assigned Accession No. ______.

Cells may be stably or transiently transformed with a vector containing the hEAAT2 promoter expression cassette as described above using methods known to those of ordinary skill in the art. Constructs containing the hEAAT2 promoter expression cassette are constructed using well-known recombinant DNA methods.

The transformed cells are contacted with the agent to be tested for its ability to modulate the transcription of the reporter gene operably linked to the hEAAT2 promoter. A detectable increase or decrease in transcription of the reporter gene is indicative of an agent that alters the activity of the hEAAT2 promoter and hence glutamate transport.

Modulation in this context is defined as an increase or decrease of at least 5% in transcription of the reporter gene in the presence of the candidate agent relative to the level of transcription in the absence of the agent. In preferred embodiments, the level of increase or decrease is greater than 10% or more preferably greater than 20%.

Agents identified by this method may be useful for the treatment of a variety of neuropathologies associated with glutamate excitotoxicity, including but not limited to damage caused by ischemia, temporal lobe epilepsy, Alzheimer's disease (AD), Huntington's disease (HD), amyotrophic lateral sclerosis (ALS) and the transmissible spongiform encephalopathies (TSEs).

The present invention further provides kits useful for identifying an agent that modulates glutamate transport. The kits may comprise a vector in which an hEAAT2 promoter nucleic acid is operably linked to a reporter gene, cells suitable for expression of the hEAAT2 promoter expression cassette contained in the vector, the reagents necessary to introduce the vector into the cells and the reagents necessary to monitor the expression of the reporter gene. Alternatively, the kits may contain cells already transformed by a vector comprising an hEAAT2 promoter expression cassette and the reagents necessary to monitor the expression of the reporter gene.

### 5.4 Identification of Agents that Modulate Signal Transduction Pathways or Other Biological Processes that Regulate Extracellular Glatamate

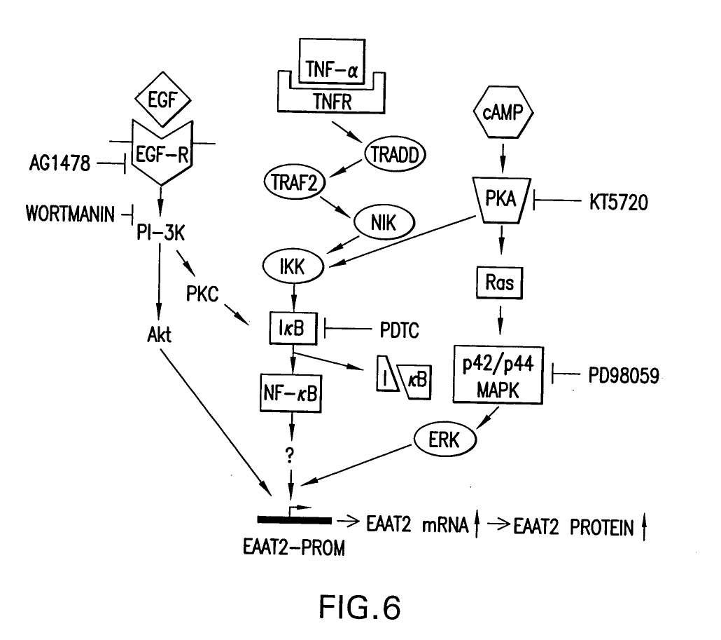

As described in the Discussion section below, the studies described in the various Examples contained herein demonstrate that multiple and converging signal transduction pathways, including those outlined in FIG. 6, are involved in the regulation of hEAAT2 promoter activity. hEAAT2 promoter activity is also downregulated by the product of the astrocyte enhanced gene 1 (AEG-1). The present invention therefore provides for the use of hEAAT2 nucleic acids to identify agents that modulate a number of different signal transduction pathways or other biological processes that may also regulate extracellular glutamate levels. In certain embodiments, these methods comprise (i) operably linking a nucleic acid sequence comprising an hEAAT2 promoter nucleic acid to a reporter gene of interest, (ii) introducing the resulting hEAAT2 promoter expression cassette into a target cell, (iii) contacting the target cell with a test agent that potentially modulates one of the signal transduction pathways or other biological processes that affect hEAAT2 promoter activity, including but not limited to those depicted in FIG. 6, and (iv) comparing the level of reporter gene expression in the presence and absence of the test agent, wherein a test agent that modulates a number of different signal transduction pathways or other biological processes that may also regulate extracellular glutamate levels is one that produces a discernible change in the level of reporter gene expression in the presence and absence of the agent. Agents that increase hEAAT2 promoter activity identified by this assay may be useful in the prevention, palliation or treatment of neurodegenerative and/or cerebrovascular diseases associated with glutamate excitotoxicity.

Cells useful for the assays of the present invention include any eukaryotic or prokaryotic cells in which the hEAAT2 promoter is active. Preferred cells include but are not limited to primary human fetal astrocytes (PHFA), PHFA cells immortalized by transformation with the human telomerase reverse transcriptase (hTERT), or H4 malignant human glioma cells. In a particular, non-limiting embodiment, the cells may be PHFA-Im cells, which are PHFA cells immortalized by transformation with hTERT, as deposited with ATCC and assigned Accession No. ______.

Cells may be stably or transiently transfected with a vector containing the hEAAT promoter expression cassette as described above using methods known to those of ordinary skill in the art. Constructs containing the hEAAT2 promoter expression cassette are created using well-known recombinant DNA methods.

The transformed cells are contacted with the agent to be tested for its ability to modulate a number of different signal transduction pathways or other biological processes that may also regulate extracellular glutamate levels by examining the affect of the agent on the transcription of a reporter gene operably linked to the hEAAT2 promoter. A detectable increase or decrease in transcription of the reporter gene is indicative of an agent that alters the activity of the hEAAT2 promoter and hence one of the various signal transduction pathways or other biological processes that may also regulate extracellular glutamate levels via their effects on the transcriptional activation of the hEAAT2 promoter. The specificity of this effect for the particular pathway or agent being examined can be confirmed through the retesting of the candidate agent in the presence and absence of known agonists, antagonists or inhibitors of components of the signal transduction pathway or other biological process being examined.

Modulation in this context is defined as an increase or decrease of at least 5% in transcription of the reporter gene in the presence of the candidate agent relative to the level of transcription in the absence of the agent. In preferred embodiments, the level of increase or decrease is greater than 10% or more preferably greater than 20%. In particularly preferred embodiments, the level of increase or decrease is greater than 50%.

Agents identified by this method may be useful for the treatment of a variety of neuropathologies in which the any of the components of the signal transduction pathways or other biological processes illustrated in FIG. 6 have been implicated, including but not limited to gliomas.

The present invention further provides kits useful for identifying an agent that modulate a number of different signal transduction pathways or other biological processes that may also regulate extracellular glutamate levels. The kits may comprise a vector in which an hEAAT2 promoter nucleic acid is operably linked to a reporter gene, cells suitable for expression of the hEAAT2 promoter expression cassette contained in the vector, the reagents necessary to introduce the vector into the cells and the reagents necessary to monitor the expression of the reporter gene. Alternatively, the kits may contain cells already transformed by a vector comprising an hEAAT2 promoter expression cassette and the reagents necessary to monitor the expression of the reporter gene.

In a first specific, non-limiting example, agents that modulate glutamate transport may be identified by 1) culturing PHFA, PHFA-Im, or H4 cells; 2) adding various concentrations of the agent to be examined or a suitable control to the cultured cells; 3) cotransfecting the cultured cells with a first plasmid containing an expression cassette comprising the hEAAT2 promoter operably linked to a first reporter gene and a second plasmid containing an expression cassette comprising a constitutive promoter such as CMV, RSV, or EF-1α, operably linked to a second reporter gene; 4) harvesting the transfected cells; 5) preparing cell lysates; 6) assaying the lysates for the presence of activity of the first and second reporter genes; and 7) normalizing the activity of the first reporter gene for variations in transfection efficiency between cell samples by dividing the activity of the first reporter gene by the activity of the second reporter gene.

In a second specific, non-limiting example, agents that modulate glutamate transport may be identified by 1) culturing PHFA, PHFA-Im, or H4 cells; 2) cotransfecting the cultured cells with a first plasmid containing an expression cassette comprising the hEAAT2 promoter operably linked to a first reporter gene and a second plasmid containing an expression cassette comprising a constitutive promoter such as CMV, RSV, or EF-1α, operably linked to a second reporter gene; 3) adding various concentrations of the agent to be examined or a suitable control to the cultured cells; 4) harvesting the transfected cells; 5) preparing cell lysates; 6) assaying the lysates for the presence of activity of the first and second reporter genes; and 7) normalizing the activity of the first reporter gene for variations in transfection efficiency between cell samples by dividing the activity of the first reporter gene by the activity of the second reporter gene.

### 5.5 hEAAT2 Promoter/mda-7 Constructs and their Uses

As noted above in Section 5.2, an hEAAT2 promoter expression cassette of the instant invention may comprise the hEAAT2 promoter operably linked to a biologically-active anti-cancer gene, including but not limited to a pro-apoptotic gene, a suicide gene, a tumor suppressor gene, a gene encoding a toxin, a gene encoding a prodrug enzyme, or any other physiologically-relevant or therapeutically desirable genes known to those of ordinary skill in the art. Such a construct, which is depicted schematically in FIG. 9, may be useful to obtain astrocyte-, neuron- or brain cell-specific gene expression. Specific expression of pro-apoptotic genes, suicide genes, tumor suppressor genes, genes encoding one or more toxins, or genes encoding a prodrug enzyme may be useful in the treatment of astrocyte-, neuron- or brain cell-specific tumors or cancers. Such cancers include, but are not limited to astrocytomas, glioblastomas, neuromas, and schwannomas. Specific expression of these genes in brain, astrocytes or neurons also may be useful in the treatment of cancers of non-neuronal or non-glial etiology that are present in the CNS (e.g. cancers of the pituitary or lymphomas) or non-neuronal or non-glial cancers that have metastasized to the CNS. Brain-, neuron- or glial-specific expression of pro-apoptotic genes, suicide genes, tumor suppressor genes, genes encoding one or more toxins, genes encoding a prodrug enzyme, or more specific approaches such as expression of gene-specific ribozymes, RNAi (interfering RNAs), triplex or antisense molecules, also may be useful in the treatment of infectious diseases in which neurons or glial cells may act as hosts for the infectious agent.

In a preferred embodiment, the anti-cancer gene to be expressed from the hEAAT2 promoter cassette is MDA-7 (IL-24). See e.g. Sarkar et al., 2002, Biotechniques (Suppl):30-39; Sauane et al., 2003, Cytokine Growth Factor Rev. 14(1):35-51; Su et al., 2003, Oncogene. 22(8):1164-1180. Expression of MDA-7 may be useful in treating both primary CNS malignancies as well as metastatic malignancies via a bystander effect. The gene encoding MDA-7, as defined herein, is: 1) a nucleic acid as set forth in SEQ ID NO:7 (GenBank Accession No. U16261; Jiang et al., 1995, Oncogene 11:2477-2486); 2) a nucleic acid that encodes MDA-7, which in specific, non-limiting embodiments is a protein having 206 amino acids with a size of 23.8 kDa and an amino acid sequence as set forth in SEQ ID NO:8 (GenBank Accession No. U16261; Jiang et al., 1995, Oncogene 11:2477-2486); or 3) functional equivalents thereof.

The mda-7 gene may be a genomic sequence containing introns but is more preferably a cDNA. The term mda-7 gene, as used herein, further encompasses nucleic acids preferably having between 400 and 2500 nucleotides, more preferably having at least 550, 600 or 650 nucleotides, which retain mda-7 function as a growth suppressant and pro-apoptotic molecule and which hybridize to a nucleic acid having a sequence as set forth in SEQ ID NO:7 under stringent hybridization conditions as set forth in “Current Protocols in Molecular Biology,” Volume 1, Ausubel et al., eds. John Wiley: New York N.Y. pp. 2.10.1-2.10.16, first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised on 40 mM NaPO₄, pH 7.2, 1-2% SDS and 1 mM EDTA.

In other preferred embodiments, mda-7 genes that hybridize under conditions of high stringency to the coding region of the nucleic acid sequence of SEQ ID NO:7 have at least about 70% sequence identity to the ooding region of the nucleic acid sequence of SEQ ID NO:7, preferably at least 75%, more preferably at least 90%, and most preferably at least 95% sequence identity to the coding region of the nucleic acid sequence of SEQ ID NO:7. The identity between two sequences is a direct function of the number of matching or identical positions. When a subunit position in both of the two sequences is occupied by the same monomeric subunit, e.g. if a given position is occupied by an adenine in each of two DNA molecules, then they are identical at that position. For example, if 7 positions in a sequence 10 nucleotides in length are identical to the corresponding positions in a second 10-nucleotide sequence, then the two sequences have 70% sequence identity. The length of comparison sequences will generally be at least 50 nucleotides, preferably at least 60 nucleotides, more preferably at least 75 nucleotides, and most preferably 100 nucleotides. Sequence identity is typically measured using sequence analysis software (e.g. Sequence Analysis Software Package of the Genetics Computer Group, University of Wisconsin Biotechnology Center, 1710 University Avenue, Madison, Wis. 53705) or other computer programs and/or algorithms known to those of ordinary skill in the art.

The term mda-7 gene as used herein further applies to nucleic acids containing terminal or internal deletions, insertions or substitutions, provided that those deletions, insertions or substitutions do not abrogate the ability of the protein encoded by the mda-7 gene to suppress the growth of or induce apoptosis or cell death in a given target cancer cell at a level relative to wild-type MDA-7 protein of at least about 10, 20, 30, 40, 50, 60, 70, 80 or 90 percent. For example, nucleic acids encoding a secreted form of MDA-7 lacking the N-terminal 48 amino acids of the coding sequence contained in SEQ ID NO:8 are known in the art (“secreted MDA-7,” “sMDA-7” or “SP MDA-7) and are also an object of the instant invention, insofar as they retain at least about 10% of wild-type MDA-7 biological activity. Nucleic acids encoding proteins lacking approximately 5, 10, 15, 20 or 25% of the N- or C-terminal amino acids of MDA-7 are also objects of the instant invention, provided that they retain at least about 10% of wild-type MDA-7 biological activity.

As used herein, “MDA-7 biological activity” is defined as the ability to suppress growth and/or induce apoptosis and/or sensitize cells to the growth-suppressive or pro-apoptotic effects of radiation in a diverse group of transformed cell types without affecting these same properties in non-transformed cell types of similar origin. Examples of MDA-7 biological activity may be found, inter alia, in Su et al., 1998, Proc. Natl. Acad. Sci. USA 95:14400-14405 (breast cancer but not normal breast tissue) or Lebedeva et al., 2002, Oncogene 21:708-718 (melanoma but not melanocytes), the contents of which are incorporated by reference herein in their entireties.

The term “MDA-7” as used herein refers to a protein encoded by a mda-7 nucleic acid as defined hereinabove. In one specific, non-limiting embodiment, MDA-7 has essentially the amino acid sequence of SEQ ID NO:8 as provided in Genbank Accession Number U16261 (“wtMDA-7”), or a functional equivalent thereof. A “functional equivalent” of the MDA-7 protein is a polypeptide whose sequence is altered by any deletion, insertion, and/or addition that does not destroy the MDA-7 biological activity of the polypeptide. “MDA-7 biological activity” is the ability to suppress growth and/or induce apoptosis and/or sensitize cells to the growth-suppressive or pro-apoptotic effects of radiation in a diverse group of transformed cell types without affecting these same properties in non-transformed cell types of similar origin. One type of functional equivalent of MDA-7 contains terminal or internal deletions, insertions or substitutions of amino acids, preferably involving up to about 1, 5, 10, 20, 25, or 30% of the total number of amino acids of the wtMDA-7 protein, provided that these deletions, insertions or substitutions do not abrogate the ability of the protein encoded by the mda-7 gene to suppress the growth of or induce apoptosis or cell death in a given target cancer cell at a level relative to wild-type MDA-7 protein of at least about 10, 20, 30, 40, 50, 60, 70, 80 or 90 percent. A specific non-limiting example of such a functional equivalent is secreted MDA-7 (“sMDA-7”), which lacks the 48 amino acids comprising the N-terminus of the MDA-7 polypeptide.

Another type of functional equivalent is MDA-7 comprised in a fusion protein. A specific, non-limiting example of a functional equivalent of wt MDA-7 is GST-MDA-7, produced by an expression system wherein MDA-7 is fused to glutathione-S-transferase. More preferably, the secretory sequence of MDA-7 is deleted in the GST-MDA-7 fusion protein.

The following nonlimiting examples serve to further illustrate the present invention.

### 6. EXAMPLE

### 6.1. Materials and Methods

Primary Cell Cultures, Cell Lines and Reagents. Primary normal human fetal astrocytes (PHFA) were isolated from second trimester (gestational age 16-19 weeks) human fetal brains obtained from elective abortions in full compliance with NIH guidelines and cultured as previously described (Bencheikh et al. 1999, J Neurovirol 5(2):115-124; Canki et al., 2001, J Virol 75(17):7925-7933; Su et al., 2002, Oncogene 21(22):3592-602). Early passage primary human mammary epithelial (HMEC) and human prostate epithelial (HPEC) cells were obtained from Clonetics Inc. (San Diego Calif.) and were cultured as described (Su et al., 1998, Proc Natl Acad Sci USA 95(24):14400-14405; Huang et al., 2001, Oncogene 20(48):7051-7063). SV40-immortalized normal human foreskin melanocyte cells (FM516-SV) and HO-1 human melanoma cells were cultured as described (Huang et al., 2001, Oncogene 20(48):7051-7063; Lebedeva et al., 2002, Oncogene 21(5):708-718). DU-145, MCF-7, Colo 205 and PANC-1 cells were from the American Type Culture Collection and maintained as described (Huang et al., 2001, Oncogene 20(48):7051-7063). Culture media and cells were tested for mycoplasma contamination using the Mycoplasma PCR ELISA kit (Roche Molecular Biochemicals, IN) and only negative cultures were used. EGF, TGF-α and TNF-α were from Invitrogen (Carlsbad Calif.), dbcAMP, bromo-cAMP, AG1478, PDTC and PD98059 were from Sigma (St. Louis Mo.) and KT5720 and wortmannin were from CalBiochem (La Jolla Calif.).

hEAAT2 Promoter Isolation. A sequential progressive genomic scanning (SPGS) cloning approach was used to identify a 5′ region upstream of the hEAAT2 cDNA containing the putative promoter region of the hEAAT2 gene. Nylon filters containing a human genomic BAC library were screened using a PCR amplified 32P-labeled exon 2 hEAAT2 (bp 105 to bp 605) probe. This screening identified three clones, FBAC-4434 BAC library, plate #354j11, 362h20, 433n05 (Incyte Genetics). All three independent BAC clones contained the hEAAT2 second exon with a large intron preceding this sequence. Probing a Southern blot containing the digested BACs with an end-labeled primer containing the first exon of hEAAT2 indicated the absence of exon 1. The 3 BACs were sequenced with T3 and T7 primers to determine the sequences in the proximity of the vector to facilitate re-screening of the library. This sequencing information permitted the generation of an intervening sequence probe that extended ˜50 kb into the first intron and resulted in the identification of 3 additional clones. Southern blotting analysis revealed that these 3 BACs contained the first hEAAT2 exon. SacII digestion (2.5 kb) of the BAC clones generated fragments containing the first exon of hEAAT2 and the 5′ upstream region. This fragment, designated as p-2426 contained the putative hEAAT2 promoter region and a part of the first exon.

Primer Extension Analysis and Nuclear Run-on Assays. Primer extension assays were performed as described (Su et al., 2000, Oncogene 19(30):3411-3421). A primer with the sequence 5′-TAATCCGCGTCCCGGCTCTCCACGGCGCGCGA-3′ (SEQ ID NO:6) complementary to the 5′ UTR of the hEAAT2 cDNA was used for this assay. Nuclear run-on assays were performed as described (Su et al., 1997, Proc Natl Acad Sci USA 94(17):9125-9130).

Construction of hEAAT2 Promoter Deletion Mutants and Performance of the Luciferase Assays. 5′-deletion mutants of the hEAAT2 promoter were made with exonuclease III digestion using the Erase-A-Base System (Promega) as described for the PEG-3 promoter (Su et al., 2000, Oncogene 19(30):3411-3421). The flhEAAT2Prom-luc and flhEAAT2Prom-luc deletion mutants were cloned into the pGL3-basic luciferase reporter vector (Promega) and luciferase reporter assays were performed as described (Su et al., 2000, Oncogene 19(30):3411-3421; Su et al., 2001, Nucleic Acids Res 29(8):1661-1671), except that instead of using lipofectamine, which was toxic to PHFA, the calcium phosphate precipitation transfection technique was used (Babiss et al., 1986, Proc Natl Acad Sci USA 83(7):2167-2171).

Northern and Western Blotting Assays. Total cellular RNA was isolated by the guanidinium/phenol extraction method and Northern blotting was performed as described (Huang et al., 2001, Oncogene 20(48):7051-7063; Su et al., 2000, Oncogene 19(30):3411-3421; Su et al., 2001, Nucleic Acids Res 29(8):1661-1671). Western blotting assays were performed as described (Su et al., 1998, Proc Natl Acad Sci USA 95(24):14400-14405; Su et al., 2002, Oncogene 21(22):3592-602).

### 6.2. Results

Cloning of the hEAAT2 Promoter Using the SPGS Cloning Approach and Identification of the hEAAT2 Transcription Start Site. A previous study of hEAAT2 structure concluded that the hEAAT2 gene region is composed of 11 exons spanning >50-Kb of genomic DNA (Meyer et al., 1998, Neurosci Lett 1998;241(1):68-70). However, despite the paramount importance of hEAAT2 regulation in normal brain function and its potential involvement in multiple neuropathologies, the structure of the hEAAT2 promoter or its role in controlling hEAAT2 expression remained unknown. The present studies provide a possible explanation for the difficulties encountered in cloning the hEAAT2 promoter. The hEAAT2 genomic region was reanalyzed and it was found that the previously proposed structure of the hEAAT2 gene (Meyer et al., 1998, Neurosci Lett 241(1):68-70) is not correct relative to the 5′ region. Current information in GenBank (Accession #Z32517) contains only a partial sequence of exon 1, consisting of 105 bp. Exon 1 is separated from exon 2 by an intron of ˜100 kb (FIG. 1C). This structure prevents a simple genomic walking approach or a single BAC library screening approach (using the previously identified 105 bp fragment) for identifying the putative 5′-region containing the hEAAT2 promoter.

To clone the hEAAT2 promoter, an ‘SPGS’ cloning strategy was employed in which nylon filters containing a human genomic BAC library were initially screened using a PCR-amplified α-[³²P]-dCTP-labeled hEAAT2 exon 2 probe. This screening identified clones containing exon 2 with a large intron preceding this sequence. Additional screening using probes containing part of the sequence of intron 1 identified three clones that contained the sequence of exon 1 and ˜2.5 kb of the 5′-upstream region. Sequence analysis of this putative hEAAT2 promoter region revealed that it contains five Sp1 sites and GC-rich repeats, but no TATA box (FIG. 1A). A similar genomic structure is found in the promoter of the ASCT1 gene, which also lacks well defined cis elements while containing five Sp1 sites and GC-rich repeats (commonly found in early growth response genes, such as those in the EGF family and jun D) (Gegelashvili et al., 1997, Mol Pharmacol 52(1):6-15). Bioinformatic analysis of the promoter region revealed a number of potential regulatory transcription factors and promoter binding elements that may contribute to hEAAT2 expression and its regulation, including N-FAT, NF-κB and N-myc (FIG. 1A).

To determine the transcriptional initiation site of the hEAAT2 gene a labeled antisense primer was hybridized to total RNA from PHFA and the extension products were separated on a sequencing gel (FIG. 1B). This experiment showed that the major transcript is being initiated from an adenosine residue located 283 bp upstream of the ATG start codon. Accordingly, this base was designated as bp +1 and extended the 5′-end of previously cloned hEAAT2 cDNA by 194 bp. These results confirm that the first exon contains 299 bp (the originally reported sequence of 105 bp and an additional 194 bp now identified by primer extension analysis) (FIGS. 1B, 1C).

Preferential Expression of the hEAAT2 Promoter in PHFA and Deletion Analysis of the hEAAT2 Promoter. hEAAT2 is expressed in brain-derived cells, mainly astrocytes (Tanaka et al., 1997, Science 276(5319):1699-1702; Anderson et al., 2000, Glia 32(1):1-14). Experiments were performed to confirm hEAAT2 promoter activity in normal human astrocytes and to determine expression levels in other cell types (FIG. 2A). Primary early passage and established human normal and tumor cell lines were co-transfected with a putative full-length ˜2.5 kb hEAAT2 promoter, a SacII fragment extending from bp −2426 to bp +44, was cloned into the pGL3-basic vector (Promega), where it drove expression of the firefly luciferase (luc) gene, and a pSV-β-galactosidase expression plasmid. Relative fold expression of a full length hEAAT2 promoter-luciferase (flhEAAT2Prom-luc) construct was determined as described (Su et al., 2000, Oncogene 19(30):3411-3421; Su et al., 2001, Nucleic Acids Res 29(8):1661-1671), and activity versus transfection with a pSV-β-galactosidase plasmid was calculated as described (Su et al., 2000, Oncogene 19(30):3411-3421; Su et al., 2001, Nucleic Acids Res 29(8):1661-1671) to equalize for differences in transfection efficiency.

The results of these studies are shown in FIG. 2A. Highest expression was consistently seen in early passage (#1 to #3) PHFA. hEAAT2 promoter activity was ˜10-fold higher in PHFA than in the other cell types analyzed (FIG. 2A). With repeated passage, hEAAT2-Prom activity decreased ˜3- to 5-fold in PHFA cells by passage #5 or #6, respectively (FIG. 2B). In contrast, negligible hEAAT2 promoter activity was apparent in additional human cells, including HMEC (passage #5), HPEC (passage #4), FM516-SV, MCF-7, DU-145, PANC-1, HO-1 and Colo 205. Elevated hEAAT2 promoter activity was found in one of six gliomas (data not shown). These results confirm preferential expression of the full-length hEAAT2 promoter in normal astrocytes.

To identify cis-acting elements important for expression of hEAAT2, a series of 5′-deletion mutants were constructed and evaluated in PHFA (FIG. 3A). Deletion of the most distal region from −2426 to −703 did not alter hEAAT2 promoter activity, suggesting that this region does not contain elements indispensable for hEAAT2 promoter activity. However, deletion of bp −703 to −326 reduced promoter activity by about ˜1.7-fold versus the putative full-length promoter or the deletion mutant ending at bp −703 (FIG. 3B). Sequence analysis of this region revealed five Sp1 binding sites and one binding site for each of the transcription factors NF-κB, N-myc and NFAT (FIG. 1A). Deletion of the region from bp −326 to bp −120 further reduced the activity of the hEAAT2 promoter by more than 2-fold (FIG. 3B). These results suggest that a putative transcription regulatory element(s) present in this region contributes to hEAAT2 promoter activity in PHFA. At present, the only recognized transcription factor-binding site present in this region is NF-κB (FIG. 1A). Further studies are required to determine to functional significance of this site for hEAAT2 promoter activity.

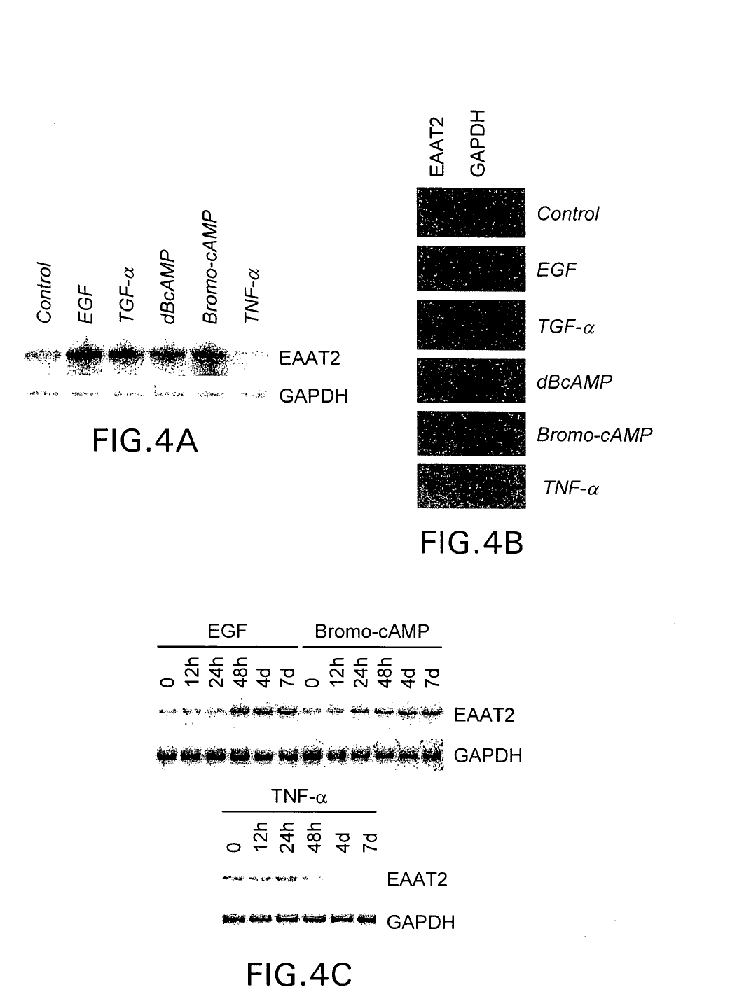

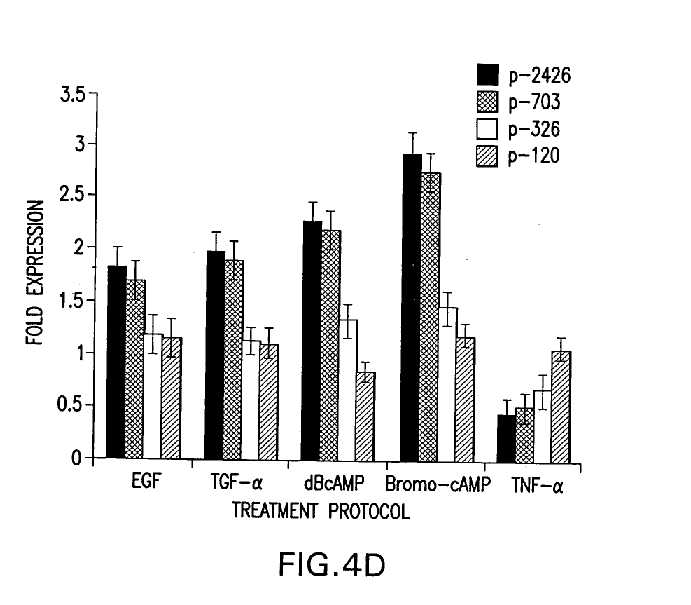

Positive and Negative Regulation of hEAAT2 Transcription, Promoter Activity and mRNA Levels in PHFA. Several enhancers of GLT-1 expression in rat astrocytes have been identified, including epidermal growth factor (EGF), transforming growth factor-α (TGF-α) and dibutyryl cyclic AMP (dbcAMP) (Swanson et al., 1997, J Neurosci 17(3):932-940; Zelenaia et al., 2000, Mol Pharmacol 57(4):667-678). Based on these considerations, experiments were performed to determine if these agents similarly modify human hEAAT2 expression in PHFA. Consistent with previous observations in rat astrocytes, 7-day treatment with EGF, TGF-α, and two analogs of cAMP, dbcAMP and bromo-cAMP, stimulated hEAAT2 mRNA expression in PHFA, whereas TNF-α decreased expression (FIG. 4B). EGF upregulated hEAAT2 mRNA to the highest extent at 48 h, and bromo-cAMP enhanced hEAAT2 mRNA expression by 24 h with a maximum level of expression observed at 48 h. In contrast, TNF-α decreased expression by 48 h (FIG. 4C).

To examine whether stimulation of hEAAT2 expression involves transcriptional changes, nuclear run-on assays were performed. As shown in FIG. 4B, the relative rate of transcription of hEAAT2 RNA, as compared with the housekeeping gene GAPDH, was elevated in PHFA following treatment with EGF, TGF-α, dbcAMP and bromo-cAMP and decreased with TNF-α treatment. These data confirm that these regulators of hEAAT2 glutamate transporter function exert their effect on steady-state mRNA by altering hEAA T2 transcription in PHFA.

To examine further the relationship between hEAAT2 and treatment with the various glutamate transporter modulators, transient transfection assays were performed in PHFA using the flhEAAT2Prom-luc constructs and various deletions thereof (FIG. 4D). Four day treatment of PHFA with EGF, TGF-α and dbcAMP resulted in ˜1.5- to 2.25-fold upregulation of hEAAT2 promoter activity. In the case of dbcAMP, promoter activity was enhanced by ˜3-fold. Deletion of the region between bp −2426 and bp −703 in the hEAAT2 promoter did not significantly decrease EGF, TGF-α, dbcAMP or bromo-cAMP stimulation suggesting that this region of the promoter does not contain transcription elements responsive to these agents. On the other hand, deletion of the region between bp −703 and bp −326 in the hEAAT2 promoter reduced EGF- and TGF-α-mediated upregulation to a level approximating that found in uninduced PHFA. A further deletion of the hEAAT2 promoter sequence between bp −326 and bp −120 did not result in any additional reduction in hEAAT2 promoter activity following EGF or TGF-α treatment. These data suggest that putative transcription regulatory motifs in the hEAAT2 promoter between bp −703 and bp −326 are determinants of elevated hEAAT2 promoter activity in PHFA following treatment with EGF or TGF-α.

In multiple experiments with different early passage PHFA, the analogs of cAMP were the most potent activators of the hEAAT2 promoter (FIG. 4). In a similar fashion as with EGF- and TGF-α-mediated hEAAT2 promoter upregulation, the region between bp −703 and bp −326 was the most relevant for dbcAMP and bromo-cAMP enhancement of promoter activity. These studies emphasize that specific regions of the hEAAT2 promoter, located predominantly between bp −703 and bp −326, contain important cis-regulatory elements that enhance promoter activity following exposure to EGF, TGF-α, dbcAMP and bromo-cAMP. In the case of TNF-α, deletion of the region of the hEAAT2 promoter between bp −326 and bp −120 resulted in promoter activity similar to that found in control untreated PHFA (FIG. 4B).

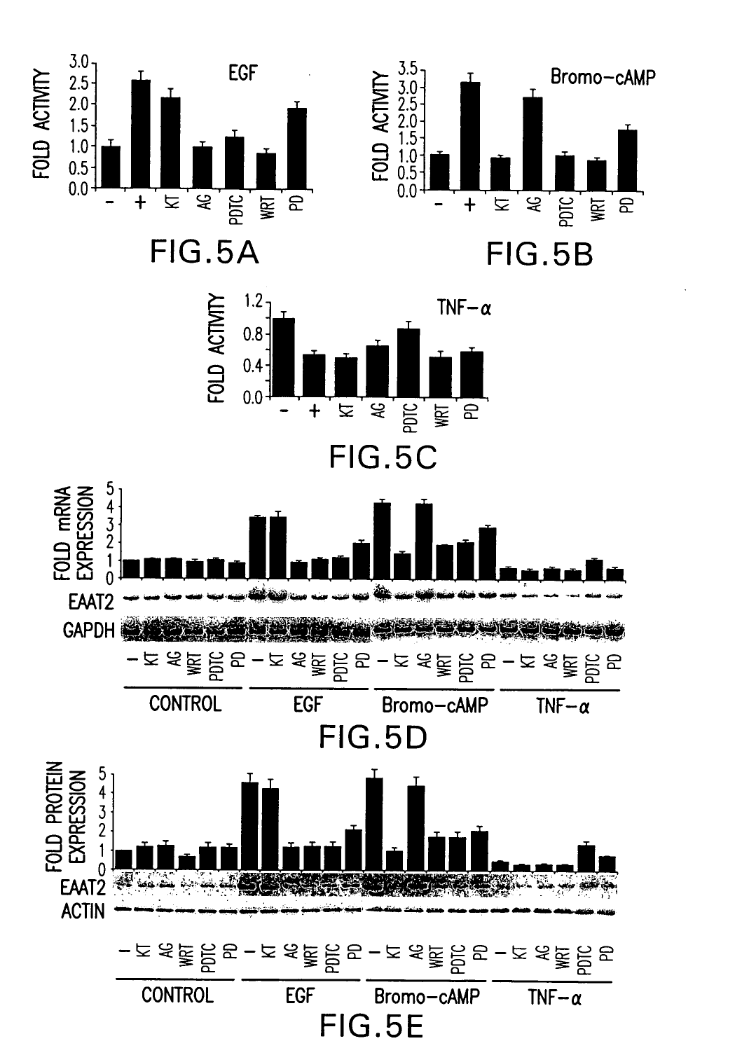

Biochemical Basis for Positive and Negative Regulation of hEAAT2 Expression in PHFA. To define the biochemical pathways relevant to the regulation of hEAAT2 expression in PHFA resulting from the different treatment protocols, a pharmacological approach was employed. This involved the use of well-characterized pathway-specific inhibitors and determining effects on hEAAT2 promoter activity, mRNA levels and protein levels (FIG. 5). The inhibitors included, KT5720 (a protein kinase A. (PKA) inhibitor), AG1478 (a tyrosine kinase inhibitor), worttnannin (a phosphatidylinositol 3-kinase (PI-3K) inhibitor), pyrrolidinedithiocarbamate (PDTC, an inhibitor of NF-κB activation) and PD98059 (a mitogen-activated kinase (MEK1/MEK2) inhibitor) (Zelenaia et al., 2000, Mol Pharmacol 57(4):667-678). In the case of EGF (or TGF-α), the enhancement of hEAAT2 promoter activity and hEAAT2 mRNA and protein levels was abolished by AG1478, PDTC and wortmannin, significantly inhibited but not extinguished by PD98059, and unaffected by KT5720 (FIGS. 5A, D and E). In the case of bromo-cAMP (or dbcAMP), the enhancement of hEAAT2 expression was eliminated by KT5720, PDTC and wortmannin, partially inhibited by PD98059, and unaffected by AG1478 (FIGS. 5B, D and E). These findings demonstrate that EGF (and TGF-α) and bromo-cAMP (and dbcAMP) enhance hEAAT2 expression through several pathways involving similar biochemical changes, both similar and distinct from each other (see FIG. 6).

Enhancement of human hEAAT2 expression by both EGF (and TGF-α) and bromo-cAMP (and dbcAMP) were inhibited by blocking NF-κB activation and PI-3K stimulation and partially inhibited by altering mitogen-activated protein kinase (MAPK, MEK1/MEK2) activation. In contrast, EGF (and TGF-α) enhancement of hEAAT2 expression involved tyrosine kinase activation and occurred in a PKA-independent manner, whereas stimulation by bromo-cAMP (and dbcAMP) was dependent on the PKA pathway but independent of tyrosine kinase activation (FIG. 6).

In contrast to the stimulatory effects of EGF (and TGF-α) and bromo-cAMP (and dbcAMP) on hEAAT2 expression, TNF-α decreased hEAAT2 expression in PHFA (FIGS. 4 and 5). Cotreatment of PHFA cells with TNF-α and the various pharmacological inhibitors demonstrated that blocking activation of the NF-κB pathway with PDTC was able to restore hEAAT2 promoter, mRNA and protein levels to that observed in untreated PHFA. A partial restoration of normal levels of hEAAT2 protein was also found in PHFA treated with TNF-α in combination with PD98059.

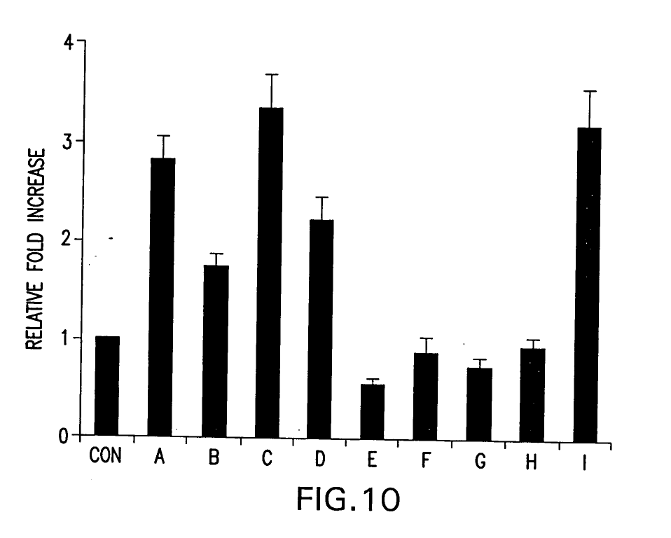

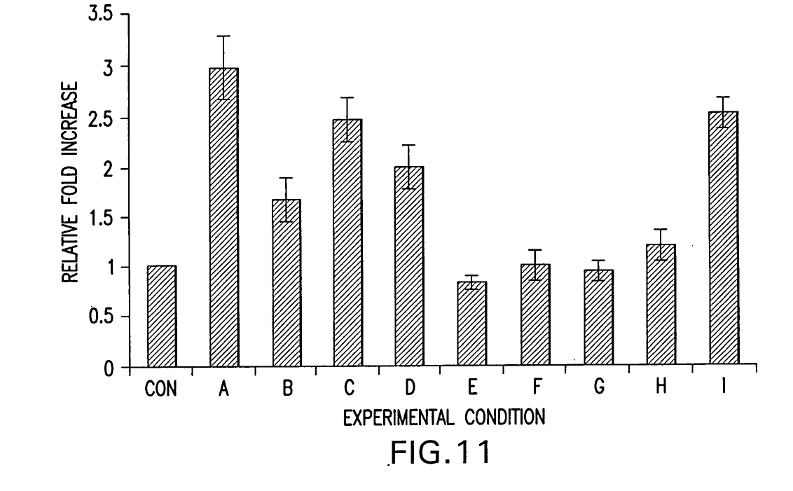

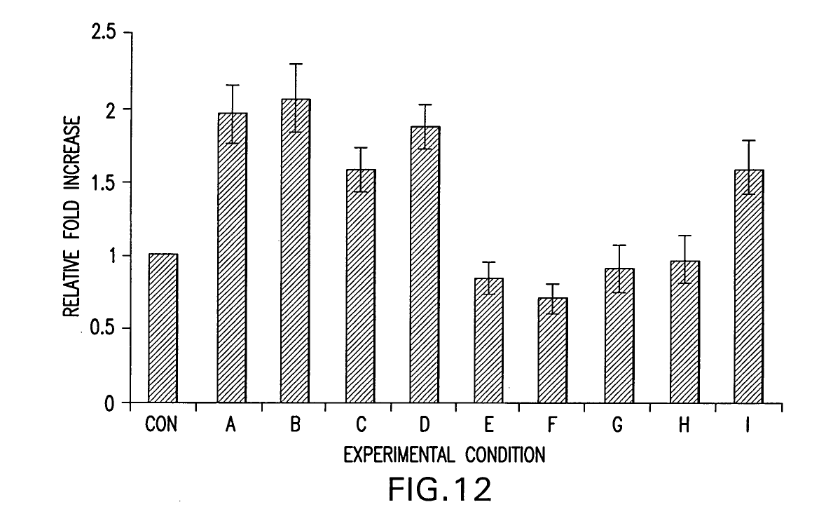

Identification of Agents that Modulate hEAAT2 Promoter Activity. FIGS. 10-12 show the effects of various agents on hEAAT2 promoter activity in PHFA (FIG. 10), PHFA-Im (FIG. 11) and H4 glioma (FIG. 12) cells. In the studies shown in FIG. 10, PHFA (passage #3) were seeded at 1×10⁵ cells/35-mm plate. Twenty-four h later cells were untreated (CON) or received the indicated compound at a final concentration of 10 μM. Forty-eight h later the cells were transfected with a FL pGL3/EAAT2 luciferase reporter construct (5 μg) plus a pSVβGalactosidase construct (1 μg) using the calcium phosphate precipitation method (Su et al. 2003, Proc. Natl. Acad. Sci. USA 100:1955-1960). After an additional 48 h, cell lysates were prepared and luciferase activity was determined using the Luciferase Assay System Kit (Promega, E1501) and luminescence determined using a luminometer (Turner Designs, TD20/20) (Su et al. 2003, Proc. Natl. Acad. Sci. USA 100:1955-1960). Data presented are the average of 3 independent plates±S.D. Qualitatively similar results were obtained in 2 additional experiments.

The effects of these same agents also were examined in two other cell lines to confirm the generality of the findings. FIG. 11 shows the results of the studies performed in PFHA-Im cells. PHFA-Im is a cell line developed from a clonal isolate of a primary human fetal astrocyte immortalized by transformation with the reverse transcriptase subunit of human telomerase. PHFA-Im cells were seeded at 5×10⁴ cells/35-mm plate. Twenty-four h later cells were untreated (CON) or received the indicated compound at a final concentration of 10 μM. Forty-eight h later the cells were transfected with a FL pGL3/EAAT2 luciferase reporter construct (5 μg) plus a pSVβGalactosidase construct (1 μg) using the calcium phosphate precipitation method (Su et al. 2003, Proc. Natl. Acad. Sci. USA 100:1955-1960). After an additional 48 h, cell lysates were prepared and luciferase activity was determined using the Luciferase Assay System Kit (Promega, E1501) and luminescence determined using a luminometer (Turner Designs, TD20/20) (Su et al. 2003, Proc. Natl. Acad. Sci. USA 100:1955-1960). Data presented are the average of 3 independent plates±S.D. Qualitatively similar results were obtained in 2 additional experiments.

H4, the third cell line examined, is a rare clone of malignant glioma cells that support hEAAT2 promoter activity. In the studies shown in FIG. 12, H4 cells were seeded at 5×10⁴ cells/35-mm plate. Twenty-four h later cells were untreated (CON) or received the indicated compound at a final concentration of 10 μM. Forty-eight h later the cells were transfected with a FL pGL3/EAAT2 luciferase reporter construct (5 μg) plus a pSVβGalactosidase construct (1 μg) using the calcium phosphate precipitation method (Su et al. 2003, Proc. Natl. Acad. Sci. USA 100:1955-1960). After an additional 48 h, cell lysates were prepared and luciferase activity was determined using the Luciferase Assay System Kit (Promega, E1501) and luminescence determined using a luminometer (Turner Designs, TD20/20) (Su et al. 2003, Proc. Natl. Acad. Sci. USA 100:1955-1960). Data presented are the average of 3 independent plates±S.D. Qualitatively similar results were obtained in 2 additional experiments.

The results of these studies identified ceftriaxone, chloramphenicol, thiamphenicol and dibutyryl cAMP as potent stimulators of hEAAT2 promoter activity in these three cell lines. These findings establish the utility of the assay system for the identification of agents which modulate hEAAT2 promoter activity. Such agents may be useful in the regulation of extracellular levels of glutamate in the central nervous system.

Use of the hEAAT2 Promoter to Produce Brain-Specific Expression of MDA-7 as a Treatment for Malignant Glioma. To determine whether expression of a secretable form of MDA-7 (SP⁻MDA-7) from astrocytes could inhibit growth of neighboring glioma cells, a replication-defective adenovirus vector was constructed in which expression of SP⁻MDA-7 (MDA-7 protein lacking the signal the 48 N-terminal amino acids that constitute the signal peptide) was under the transcriptional control of the hEAAT2 promoter of the instant invention (Ad.hEAAT2-SP⁻MDA-7). PHFA cells were transduced with Ad.hEAAT2-SP⁻MDA-7 or a control adenovirus vector, co-cultured with U251 glioma cells, and overlaid with agar, which permitted the formation of U251 colonies to be observed. detection. Transduction by Ad.hEAAT2-SP⁻MDA-7 markedly reduced U251 colony formation relative to control levels, indicating that secretion of MDA-7/IL-24 from the transduced PHFA cells inhibited growth of the glioma cells. These results suggest that secretion of MDA-7/IL-24 from cells of the CNS may be useful in treating primary or metastatic tumors present in this tissue.

### 6.2. Discussion

The foregoing examples demonstrate that multiple and converging signal transduction pathways such as those outlined in FIG. 6 are involved in regulating hEAAT2 expression in PHFA, and this regulation occurs at a transcriptional level. Expression of hEAAT2 mRNA and protein is temporally upregulated in PHFA cells following treatment with EGF, TGF-α and cAMP analogs (dbcAMP and bromo-cAMP). Using a series of pharmacological inhibitors of defined biochemical pathways (FIG. 6), the role of multiple signaling events that impinge on hEAAT2 promoter activity in PHFA has been demonstrated (FIGS. 4 and 5).

In the case of EGF and TGF-α, signaling through the EGFR and activation of PI-3K and NF-κB are primary mediators of elevated hEAAT2 expression. In the case of dbcAMP and bromo-cAMP, signaling through PKA is a major mediator of activity, and regulation of hEAAT2 expression is also exerted by PI-3K and NF-κB. Cocultivation of neurons, or neuronal conditioned medium, with rat astrocytes stimulates GLT-1 expression (Zelenaia et al., 2000, Mol Pharmacol 57(4):667-678). Similarly, rat neuronal conditioned medium also enhances human hEAAT2 expression in PHFA, suggesting that the human model is behaving in a similar manner as the rodent astrocyte model and factors regulating activity are not species specific.

Since the rat GLT-1 promoter was not available and because actinomycin D (which inhibits transcription) was toxic, it was not previously possible to determine the mechanism, i.e. activation of gene. transcription or increase in mRNA stability, involved in the increase in mRNA in rat astrocytes following treatment with EGF, TGF-α and dbcAMP (Zelenaia et al., 2000, Mol Pharmacol 57(4):667-678). The present results of nuclear run-on and promoter-based reporter assays demonstrate that these modulators of rat GLT-1 expression can exert their effects in PHFA by altering transcription of the hEAAT2 gene. Deletion analysis suggests that sequences located between bp −703 and bp −326 and between bp −326 and bp −120 in the hEAAT2 promoter may be significant targets for this regulation.

TNF-α inhibits glutamate uptake by PHFA (Fine et al., 1996, J Biol Chem 271(26):15303-15306). This inhibition of glutamate transport by TNF-α was dose-dependent and very specific, since neutralizing antibody to TNF-α abolished this inhibition and a monoclonal antibody that is an agonist at the 55-kDa TNF receptor induced inhibition (Fine et al., 1996, J Biol Chem 271(26):15303-15306). Infection of PHFA by HIV-1 or exposure of the cells to gp120 induced rapid and sustained inhibition of glutamate uptake by astrocytes and this effect correlated with a decrease in the expression of hEAAT2 protein and RNA. Consistent with this effect, exposure of PHFA to HIV-1 or gp120 decreases hEAAT2 promoter activity in these cells. These findings suggest that HIV-1, gp120, and other neuropathogenic agents can alter specific signaling pathways in astrocytes in a way that may impair important physiological functions of these cells in neuronal signal transmission and response to brain injury.

In the experiments discussed herein, TNF-α inhibited hEAAT2 RNA transcription (nuclear run-on) and promoter activity and decreased the levels of hEAAT2 mRNA and protein in PHFA cells (FIGS. 4 and 5). This effect could be reversed by simultaneous treatment with pyrrolidinedithiocarbamate (PDTC), an inhibitor of NF-κB activation (FIG. 5). Moreover, the stimulatory effect of EGF, TGF-α, dbcAMP and bromo-cAMP were also inhibited by PDTC, suggesting that NF-κB activation may be a primary contributor, acting both positively and negatively depending on the agent administered, in regulating hEAAT2 expression in PHFA. These results are consistent with a control mechanism wherein TNF-α decreases hEAAT2 activity in PHFA by decreasing the transcription, steady state-mRNA and protein levels of hEAAT2.

Cell line PHFA-In was deposited with the American Type Culture Collection (ATCC), P.O. Box 1549, Manassas Va. 20108, on Feb. 6, 2004, and assigned Accession Number ______.

Various publications are cited herein, the contents of which are incorporated by reference in their entireties.

1. An isolated nucleic acid comprising a human Excitatory Amino Acid Transporter-2 Gene (hEAA T2) promoter.

2. The isolated nucleic acid of claim 1, wherein the hEAAT2 promoter comprises the nucleic acid sequence of SEQ ID NO:1.

3. The isolated nucleic acid of claim 1, wherein the hEAAT2 promoter comprises the nucleic acid sequence of SEQ ID NO:2.

4. The isolated nucleic acid of claim 1, wherein the hEAAT2 promoter comprises the nucleic acid sequence of SEQ ID NO:3.

5. The isolated nucleic acid of claim 1, wherein the hEAAT2 promoter comprises the nucleic acid sequence of SEQ ID NO:4.

6. The isolated nucleic acid of claim 1, wherein the hEAAT2 promoter comprises the nucleic acid sequence of SEQ ID NO:5.

7. An isolated nucleic acid that hybridizes to the isolated nucleic acid of claim 1 under stringent hybridization conditions.

8. An isolated nucleic acid that is homologous and functionally equivalent to the hEAAT2 promoter.

9. A vector comprising the isolated nucleic acid of claim 1.

10. A cell comprising the vector of claim 9.

11. The cell of claim 10, wherein the cell is a primary human fetal astrocyte (PHFA) cell.

12. The cell of claim 10, wherein the cell is an immortalized primary human fetal astrocyte (PHFA-Im) cell.

13. The cell of claim 11, wherein the PHFA-Im cell is the cell line deposited with the American Type Culture Collection under ATCC Accession Number PTA-5804.

14. The cell of claim 10, wherein the cell is an H4 human glioma cell.

15. A method for achieving astrocyte-specific gene expression comprising:
(i) operatively linking the isolated nucleic acid of claim 8 with a desired gene of interest; and
(ii) introducing the resulting expression cassette into an astrocyte where astrocyte-specific gene expression is desired.

16. The method of claim 15, wherein said gene of interest is selected from a group consisting of a reporter gene or a biologically-active gene.

17. The method of claim 16, wherein said reporter gene is selected from the group consisting of a β-galactosidase gene, a β-glucuronidase gene, a β-lactamase gene, an alkaline phosphatase gene, a gene encoding secreted alkaline phosphatase, a chloramphenicol aminotransferase gene, a luciferase gene, and a gene encoding a fluorescent protein.

18. The method of claim 16, wherein said biologically-active gene is selected from a group consisting of a pro-apoptotic gene, an anti-apoptotic gene, a suicide gene, a tumor suppressor gene, a gene encoding a receptor, a gene encoding an ion channel, a gene encoding a ribozyme, a gene encoding an oligonucleotide capable of acting as an antisense or triplex reagent for gene silencing or RNA interference, a gene encoding a toxin, a gene encoding a prodrug enzyme, and a gene encoding a growth factor.

19. A method for achieving neuron-specific gene expression comprising:
(i) operatively linking the isolated nucleic acid of claim 8 with a desired gene of interest; and
(ii) introducing the resulting expression cassette into a neuron where neuron-specific gene expression is desired.

20. The method of claim 19, wherein said gene of interest is selected from a group consisting of a reporter gene or a biologically-active gene.

21. The method of claim 20, wherein said reporter gene is selected from the group consisting of a β-galactosidase gene, a β-glucuronidase gene, a β-lactamase gene, an alkaline phosphatase gene, a gene encoding secreted alkaline phosphatase, a chloramphenicol aminotransferase gene, a luciferase gene, and a gene encoding a fluorescent protein.

22. The method of claim 20, wherein said biologically-active gene is selected from a group consisting of a pro-apoptotic gene, an anti-apoptotic gene, a suicide gene, a tumor suppressor gene, a gene encoding a receptor, a gene encoding an ion channel, a gene encoding a ribozyme, a gene encoding an oligonucleotide capable of acting as an antisense or triplex reagent for gene silencing or RNA interference, a gene encoding a toxin, a gene encoding a prodrug enzyme, and a gene encoding a growth factor.

23. A method for achieving brain cell-specific gene expression comprising:
(i) operatively linking the isolated nucleic acid of claim 8 with a desired gene of interest; and
(ii) introducing the resulting expression cassette into a brain cell where brain cell-specific gene expression is desired.

24. The method of claim 23, wherein said gene of interest is selected from a group consisting of a reporter gene or a biologically-active gene.

25. The method of claim 24, wherein said reporter gene is selected from the group consisting of a β-galactosidase gene, a β-glucuronidase gene, a β-lactamase gene, an alkaline phosphatase gene, a gene encoding secreted alkaline phosphatase, a chloramphenicol aminotransferase gene, a luciferase gene, and a gene encoding a fluorescent protein.

26. The method of claim 24, wherein said biologically-active gene is selected from a group consisting of a pro-apoptotic gene, an anti-apoptotic gene, a suicide gene, a tumor suppressor gene, a gene encoding a receptor, a gene encoding an ion channel, a gene encoding a ribozyme, a gene encoding an oligonucleotide capable of acting as an antisense or triplex reagent for gene silencing or RNA interference, a gene encoding a toxin, a gene encoding a prodrug enzyme, and a gene encoding a growth factor.

27. A method for identifying an agent that modulates glutamate transport comprising:
(i) operatively linking the isolated nucleic acid of claim 8 with a reporter gene of interest;
(ii) introducing the resulting expression cassette into a target cell;
(iii) contacting the target cell with a candidate agent; and
(iv) comparing the level of reporter gene expression in the presence and absence of the agent,
wherein an agent that modulates glutamate transport is one that produces a measurable change in the level of reporter gene expression in the presence and absence of the agent.

28. The method of claim 27, wherein said reporter gene of interest is selected from a group consisting of a β-galactosidase gene, a β-glucuronidase gene, a β-lactamase gene, an alkaline phosphatase gene, a gene encoding secreted alkaline phosphatase, a chloramphenicol aminotransferase gene, a luciferase gene, and a gene encoding a fluorescent protein.

29. The method of claim 28, wherein said target cell is selected from the group consisting of a primary human fetal astrocyte (PHFA) cell, an immortalized PHFA cell, and an H4 human glioma cell.

30. A method for identifying an agent that modulates a signal transduction pathways or other biological process that regulates extracellular glutamate levels comprising:
(i) operatively linking the isolated nucleic acid of claim 8 with a reporter gene of interest;
(ii) introducing the resulting expression cassette into a target cell;
(iii) contacting the target cell with a candidate agent; and
(iv) comparing the level of reporter gene expression in the presence and absence of the agent,
wherein an agent that modulates a signal transduction pathways or other biological process that regulates extracellular glutamate levels selected from the group consisting of the cellular activity of the EGF receptor, the cellular levels of the EGF receptor, the cellular activity of the TGF-α receptor, the cellular levels of the TGF-α receptor, the cellular activity of the TNF-α receptor, the cellular levels of the TNF-α receptor, the intracellular levels of cAMP, the intracellular levels of PI-3K; the intracellular levels of PKC, the intracellular levels of Akt, the intracellular levels of TRADD, the intracellular levels of TRAF2, the intracellular levels of NIK, the intracellular levels of IKK, the intracellular levels of IκB, the intracellular levels of NF-κB, the intracellular levels of PKA, the intracellular levels of MAPK, the intracellular levels of ERK, and the intracellular levels of the ras oncogene protein is one that produces a discernible increase in the level of reporter gene expression in the presence and absence of the candidate agent.

31. The method of claim 30, wherein said reporter gene of interest is selected from a group consisting of a β-galactosidase gene, a β-glucuronidase gene, a β-lactamase gene, an alkaline phosphatase gene, a gene encoding secreted alkaline phosphatase, a chloramphenicol aminotransferase gene, a luciferase gene, and a gene encoding a fluorescent protein.

32. The method of claim 30 wherein said target cell is selected from the group consisting of a primary human fetal astrocyte (PHFA) cell, an immortalized PHFA cell, and an H4 human glioma cell.

33. A method of treating a malignancy in the central nervous system comprising introducing into the central nervous system a nucleic acid comprising an mda-7 gene operably linked to a hEAA T2 promoter.

34. Use of a composition comprising a nucleic acid comprising an mda-7 gene operably linked to a hEAAT2 promoter in the manufacture of a medicament for the treatment of a malgnancy in the central nervous system.
